(19) United States
(12) Patent Application Publication
(10) Pub. No.: US 2023/0330034 A1
(43) Pub. Date: Oct. 19, 2023

(54) COVALENTLY CROSS-LINKED GLYCOSYLATED MUCIN NANOPARTICLES AS SYSTEMS FOR THE DELIVERY AND RELEASE OF ACTIVE INGREDIENTS AND BIOMOLECULES

(71) Applicants: UNIVERSITA’ DEGLI STUDI DI TORINO, TORINO (IT); UNIVERSITA’ DEGLI STUDI DI PAVIA, PAVIA (IT); POLITECNICO DI MILANO, MILANO (IT)

(72) Inventors: Sonia Visentin, Torino (IT); Francesco Bracotti, Condove (Torino) (IT); Livia Visai, Rosate (Milano) (IT); Paola Petrini, Milano (IT)

(73) Assignees: Universita' degli Studi di Torino (80%), Torino (IT); POLITECNICO DI MILANO (10%), Milano (IT); Universita' Degli Studi di Pavia (10%), Pavia (IT)

(21) Appl. No.: 18/002,897

(22) PCT Filed: Jun. 21, 2021
(86) PCT No.: PCT/IB2021/055450

§ 371 Date: Dec. 22, 2022

(30) Foreign Application Priority Data
Jun. 22, 2020 (IT) 102020000014908

(51) Int. Cl. A61K 9/51

(52) U.S. Cl. CPC A61K 9/5169; B82Y 5/00

(57) ABSTRACT

The present invention relates to covalently cross-linked glycosylated mucin nanoparticles and the use thereof for the delivery and release of active ingredients, markers and/or biomolecules. The subject matter of the invention also relates to covalently cross-linked glycosylated mucin nanoparticles comprising at least one compound selected from an active ingredient, a marker and a biomolecule. The invention further relates to a method for preparing covalently cross-linked glycosylated mucin nanoparticles, optionally comprising at least one compound selected from an active ingredient, a marker and a biomolecule.

### TECHNICAL FIELD

The present invention relates to covalently cross-linked glycosylated mucin nanoparticles, the use thereof as a medicament and, preferably, as antivirals and the use thereof for the delivery and release of active ingredients, markers and/or biomolecules. The invention also relates to covalently cross-linked glycosylated mucin nanoparticles comprising at least one compound selected from an active ingredient, a marker and a biomolecule.

The invention further relates to a method for preparing covalently cross-linked glycosylated mucin nanoparticles, optionally comprising at least one compound selected from an active ingredient, a marker and a biomolecule.

### Prior Art

Pharmaceutical technology is confronted with the difficulty tied to the handling of high molecular weight molecules, which become very viscous and thus difficult to administer orally, by inhalation and/or by injection.

By way of example, glycomimetic drugs, i.e. carbohydrate-based drugs, pose problems from a pharmacokinetic standpoint precisely due to the presence of carbohydrates, which, being highly polar (high density of polar groups and high hydrophilicity) are absorbed orally to a limited degree (low bioavailability). Furthermore, even if they are administered by parenteral injection, the glycomimetic drugs known to date are quickly eliminated through the kidneys.

Some cases of success of known glycomimetic drugs are oseltamivir and zanamivir (Relenza), which inhibit viral neuraminidase in the treatment of influenza and molecules that promote the inhibition of viral adhesion to the epithelium. However, carbohydrates are too hydrophilic to have good bioavailability and the lability of the glycosidic bonds of glycosidase in vivo limit their application from a pharmaceutical viewpoint.

In order to overcome these limits, it would be necessary to develop glycomimetic nanosystems endowed with chemical and metabolic stability and capable of mimicking the biological activity of specific oligosaccharides.

Although glycosylated nanosystems are not present in clinical practice, various examples of glycosylated nanoparticles exist in the literature and the most widely used nanomaterials are metal nanoparticles, carbon nanotubes, liposomes and dendrimers (for example, Kottari N. et al., “Applications of Glyconanoparticles as ‘Sweet’ Glycobiological Therapeutics and Diagnostics”. Adv. Polym. Sci. (2013), vol 254, p. 297-342).

There is thus still a strongly felt need for new glycomimetic nanosystems that can serve as effective vehicles for the absorption of glycomimetic drugs.

Another very real problem in the pharmaceutical industry is resistance to antibiotics.

Antimicrobial resistance (AMR) threatens the prevention and effective treatment of an increasingly wide range of infections caused by bacteria.

In recent years, the phenomenon of antibiotic resistance has reached proportions such as constitute one of the main global public health issues. Development of resistance to all classes of antibiotics introduced up to now has been observed in clinical practice and we are witnessing the emergence of a growing number of multidrug-resistant bacteria. The identification of innovative antimicrobial agents, alongside a correct management of already existing ones, thus appears to be of primary importance. In this context, however, the big pharmaceutical companies are abandoning the development of new antimicrobials due to the considerable development costs, which are not matched by subsequent earnings, as the new antimicrobials placed on the market rapidly lose their effectiveness due to the development of phenomena of antibiotic resistance, thus reducing revenue.

The problem of bacterial infections is also tied to the formation of so-called bacterial biofilms, which makes the antibiotic activity of the known drugs even more difficult.

Mucins are high molecular weight glycoproteins capable of taking on an extended conformation and assembling into a hydrogel protecting the mucosal epithelium. A layer of mucus, several hundred micrometres thick, is obtained in nature by expansion of the mucins previously condensed inside vesicles in the cells of the mucosal epithelium, which are released outside the cells as needed.

Mucins are further capable of providing biochemical signals both to bacteria and to mammal cells, thanks to the presence of oligosaccharides (glycosylation) which recognise a class of protein receptors called lectins. Lectins control the initial stages of many infections (host-pathogen interaction) (Daniel Passos da Silva et al., Nature Communication (2019) 10:2183, https://doi.org/10.1038/s41467-019-10201-4) and may thus be considered as potential therapeutic targets, above all in the case of bacterial infections.

In fact, lectins, and in particular the lectin of Pseudomonas aeruginosa, are involved in the formation and stabilisation of bacterial biofilm.

The interaction between mucin and lectins is also important in the field of antitumour therapy (Hassan Lemjabbar-Alaouil et al. in Advances in Cancer Research, (2015) 126:305-344. doi:10.1016/bs.acr.2014.11.007).

The composition of the oligosaccharide part of mucins also includes sialic acid, where said term indicates the N- and O-substituted derivatives of neuraminic acid, i.e. a monosaccharide and 9 carbon atoms. In particular, N-acetylneuraminic acid is common.

Sialic acid bonded to glycoproteins and gangliosides is used by many viruses as a receptor for entry into human and animal cells. These viruses include important human and animal pathogenic agents, such as influenza viruses, parainfluenza viruses, mumps virus, coronaviruses, noroviruses, rotaviruses and DNA tumour viruses.

Therefore, the possibility of delivering antiviral active ingredients, such as, for example, retroviral drugs, by means of stable mucin nanoparticles that can interact with viruses thanks to the presence of sialic acid is of particular interest.

In Yan H. et al. “Reversible Condensation of Mucins into nanoparticles”, Langmuir (2018), vol. 34, p. 13615-13625 a reversible in vitro process for condensing mucins into nanoparticles by adding glycerol to an aqueous solvent in which there are purified mucins is described. Reversible aggregates are formed with this process thanks to interactions of a physical type. The authors further indicate the possibility of a partial stabilisation with calcium and polylysine, which leads, however, to the formation of reversible, unstable nanoparticles, since the interactions that occur between mucin and calcium and polylysine are of an ionic, noncovalent type.

In “Protein nanoparticles as drug delivery carriers for cancer therapy”, Lohcharoenkal W. et al., in BioMed Research International (2014), Article ID 180549, the authors indicate the preparation of protein nanoparticles, in particular based on albumin, which is a protein with a very low molecular weight compared to mucin and is not a glycoprotein.

Various formulations comprising albumin nanoparticles are available today. For example, paclitaxel adsorbed onto albumin nanoparticles is a drug approved by the FDA.

The synthesis of albumin nanoparticles is also described in Kimura et al., Chem. Pharm. Bull., (2018) vol 66, 382-390 (2018), where desolvation in ethanol followed by cross-linking with glutaraldehyde is used to obtain nanoparticles optionally loaded with anthracycline derivatives.

Notwithstanding the presence of several studies on the preparation of mucin or albumin nanoparticles, no effective system for delivering active ingredients, markers or biomolecules has yet been made available, above all in terms of overcoming the problems discussed here in relation to the bioavailability of glycomimetic drugs when administered orally or by injection and antibiotic resistance.

### Definitions

Unless defined otherwise, all of the terms of the art, notations and other scientific terms used here are intended to have the meanings commonly understood by the persons skilled in the art to which this description pertains. In some cases, terms with commonly understood meanings are defined here for the sake of clarity and/or for easy reference; the inclusion of such definitions in the present description must thus not be interpreted as indicative of a substantial difference from what is generally understood in the art.

The terms “comprising”, “having”, “including” and “containing” are to be understood as open terms (i.e. the meaning “comprising, but not limited to”) and are to be considered as a support also for terms such as “consist essentially of”, “consisting essentially of”, “consist of” or “consisting of”.

“Nanoparticles” preferably means particles with a size equal to and/or comprised between 100 and 200 nm.

“Glycomimetic drugs” means drugs in which the active ingredients (or molecules of varying nature) are combined with one or more molecules of monosaccharides, in particular with glucose molecules.

“Glycosylation” means the process of combining one or more molecules of glucose (or of other monosaccharides) with molecules of a different nature (which are thus glycosylated).

The term “sialic acid” means the N- and O-substituted derivatives of neuraminic acid, i.e. a monosaccharide and 9 carbon atoms. N-acetylneuraminic acid is preferred in particular.

“Parenterally” or “by parenteral injection” refers to the intravenous, intramuscular, subcutaneous, intraarterial, intraarticular, intrasynovial, intracardiac and intrathecal routes of administration.

“By inhalation” or “by inhalational administration” means a method of administration through the upper breathing passages, trachea and bronchi, until reaching the alveoli.

The term “molecular imaging” means the visualisation, characterisation and measurement of biological processes at a molecular or cellular level in humans or in other living organisms.

The term “one pot” refers to two or more consecutive reactions without isolation of the respective intermediate product or products.

The term “physiologically acceptable excipient” refers to a substance devoid of any pharmacological effect of its own and which does not produce adverse reactions when administered to a mammal, preferably to a human being. Physiologically acceptable excipients are well known in the art and are described, for example, in the Handbook of Pharmaceutical Excipients, sixth edition (2009), incorporated herein by reference.

The term “composition” as used in the present document is understood to include a product comprising the specified ingredients in the specified amounts, as well as any product that results, directly or indirectly, from the combination of the specified ingredients in the specified amounts. “Pharmaceutically acceptable” means that the carrier, the diluent or the excipient must be compatible with the other components of the formulation and not harmful to the recipient.

The acronym “PGM” stands for “porcine gastric mucin”.

The acronym “NP” stands for “nanoparticle”.

The acronym “NPs” stands for “nanoparticles”.

The acronym “NPs-MUCGli” stands for the covalently cross-linked mucin nanoparticles of the invention, preferably porcine gastric mucin or bovine submaxillary mucin nanoparticles.

The acronym “NPs-MUCGli/cipro” stands for the covalently cross-linked mucin nanoparticles of the invention, preferably porcine gastric mucin or bovine submaxillary mucin nanoparticles, comprising ciprofloxacin.

The acronyms “NPs-MUCGli/remd”, “NPs-MUCGli/camo”, “NPs-MUCGli/prala”, “NPs-MUCGli/RSV”, “NPs-MUCGli/doxo”, “NPs-MUCGli/trame”, “NPs-MUCGli/cy5.5”, “NPs-MUCGli/cefta”, “NPs-MUCGli/azi”, “NPs-MUCGli/desa” and “NPs-MUCGli/bari” stand for the covalently cross-linked mucin nanoparticles of the invention, preferably porcine gastric mucin or bovine submaxillary mucin nanoparticles comprising remdesivir, camostat, pralatrexate, RSV 604, doxorubicin, trametinib, cyanine 5.5, ceftazidime, azithromycin, dexamethasone and baricitinib, respectively.

The acronym “NPs-MUCGli/alb-FITC” stands for covalently cross-linked glycosylated porcine gastric mucin nanoparticles loaded with FITC-albumin.

The acronym “NPs-MUCGli/PNA-FITC”, stands for covalently cross-linked glycosylated porcine gastric mucin nanoparticles loaded with an oligonucleotide.

The acronym “PGM Yan” stands for the nanoparticle aggregates obtained in Yan H. et al. “Reversible Condensation of Mucins into Nanoparticles”, Langmuir (2018), vol. 34, p. 13615-13625, starting from native porcine gastric mucin, i.e., directly extracted.

The acronym “NPs PGM YAN” or “PGM Yan NPs” stands for the nanoparticles obtained under the optimal conditions described in Yan H. et al. “Reversible Condensation of Mucins into Nanoparticles”, Langmuir (2018), vol. 34, p. 13615-13625 (glycerol 30% v/v/H₂O), using commercial porcine gastric mucin type III instead of the directly extracted native mucin used by Yan.

“Biomolecules” means nucleic acids, peptides, lipids and growth factors.

“Markers” means fluorophores, such as, for example, fluorescein isothiocyanate, rose bengal and near-infrared fluorophores such as cyanines.

“Mucin type II” means a preparation of raw porcine gastric mucin, for example product no. M2378 in the Merck 2020 catalogue (CAS no. 84082-64-4).

“Mucin type III” means a partially purified porcine gastric mucin powder prepared according to the method described in Glenister, D. A. and Salamon, K. Microbial Ecology in Health & Disease 1, 31, (1988) This mucin is for example product no. M1778 in the Merck 2020 catalogue (CAS no. 84082-64-4).

The acronym “BSM” stands for bovine submaxillary mucin, product no. M3895 in the Merck 2020 catalogue (CAS no. 84195-52-8).

“Cross-linker” means “cross-linking agent”.

The term “[mucin]” means “concentration of mucin”.

The term “[ciprofloxacin]” means “concentration of ciprofloxacin”.

The acronym “PBS” stands for phosphate buffered saline.

The acronym “FITC” stands for the compound “fluorescein isothiocyanate”.

The acronym “NPs-MUCGli/FITC” stands for the covalently cross-linked glycosylated porcine gastric mucin nanoparticles of the invention comprising fluorescein isothiocyanate.

The acronym “PAMPA” refers to the: “parallel artificial membrane permeability assay”.

### Object of the Invention

In a first aspect the invention relates to covalently cross-linked glycosylated mucin nanoparticles optionally comprising at least one compound selected from an active ingredient, a marker and a biomolecule, wherein the nanoparticles have mucin oligosaccharide chains, i.e. the glycosylated part of the mucin, on the surface of the nanoparticles.

According to a preferred aspect, the mucin used for the covalently cross-linked glycosylated nanoparticles of the invention is porcine gastric mucin (PGM) or bovine submaxillary mucin (BSM). According to an aspect further preferred, the porcine gastric mucin is porcine gastric mucin type III.

The nanoparticles of the invention are used alone as a medicament, preferably as antivirals, or for the delivery and the release of active ingredients, markers and/or biomolecules. Preferably, the active ingredients are antibiotics and the nanoparticles thus loaded are capable of delivering the drugs towards bacteria and/or the bacterial biofilm, thanks to the bonding between the lectins present in the bacteria and the oligosaccharides of the glycosidic part of the mucin arranged on the surface of the nanoparticles.

According to a further preferred aspect, the active ingredients are antivirals and the nanoparticles of the invention thus loaded are capable of delivering the drugs towards viruses, possibly also in the presence of mucus, thanks to their mucoadhesive characteristics and the bonding of the viruses with the sialic acids and lectins present in the oligosaccharide chains of the surface glycosylation of the nanoparticles.

When they are not loaded with active ingredients, the nanoparticles of the invention can inhibit the interaction of viruses with some cellular receptors through a competition mechanism; for example, they can inhibit the interaction between the virus SARS-CoV-2 and ACE2 receptors (“Receptor recognition by novel coronavirus from Wuhan: an analysis based on decade-long structural studies of SARS”, Yushun Wan, Jian Shang, Rachel Graham, Ralph S Baric, Fang Li. Journal of Virology, 2020; DOI: 10.1128/JVI.00127-20). Therefore, the nanoparticles of the invention not loaded with active ingredients show antiviral activity.

If loaded with one or more antiviral active ingredients, the nanoparticles of the invention show a synergy of action towards viruses because they can release the antiviral active ingredient delivered and simultaneously inhibit the viruses through competition at their receptor sites. The invention also relates to a process for preparing covalently cross-linked glycosylated mucin nanoparticles which enables the nanoparticles to be obtained with a “one-pot” reaction.

### BRIEF DESCRIPTION OF THE DRAWINGS

The invention will be described below with reference to some examples provided for non-limiting explanatory purposes and illustrated in the appended figures.

FIG. 1A shows the UV-visible (UV-vis) spectrum of the NPs-MUCGli obtained in example 1 compared to the UV-vis spectrum of PGM alone, FIG. 1B shows a graphic exemplification of the NPs-MUCGli obtained, in which it is possible to observe the arrangement of the oligosaccharide chains on the surface of the nanoparticles.

FIG. 2A shows the TEM (transmission electron microscope) images and in FIG. 2B it is possible to see the EDS X-ray analysis of the NPs-MUCGli obtained in example 1.

FIG. 3 shows the analysis of the NPs-MUCGli obtained in example 1, performed with the DLS (Dynamic Light Scattering) technique.

FIG. 4 shows the UV-visible (UV-vis) spectrum of the NPs-MUCGli/cipro obtained in example 2 compared to the UV-vis spectrum of PGM alone and ciprofloxacin alone.

FIG. 5 shows the TEM (transmission electron microscope) image of the NPs-MUCGli obtained in example 2.

FIG. 6 shows the UV-vis spectra that represent the release of ciprofloxacin over time by NPs-MUCGli/cipro.

FIG. 7 shows the UV-vis spectra of the NPs-MUCGli/FITC obtained in example 3, of FITC, of PGM and of NPs-MUCGli and NPs-MUCGli/FITC.

FIG. 8 shows the TEM (transmission electron microscope) images of the NPs-MUCGli/FITC obtained in example 3.

FIG. 9 shows the calibration curves of ciprofloxacin (9A) and propranolol (9B) for permeability studies on NPs-MUCGli/cipro (PAMPA) in the absence and presence of a mucus model (as described in Pacheco, D. P., Butnarasu, C. S., Briatico Vangosa, F., Pastorino, L., Visai, L., Visentin, S., Petrini, P. “Disassembling the complexity of mucus barriers to develop a fast screening tool for early drug discovery” (2019) Journal of Materials Chemistry B, 7 (32), pp. 4940-4952).

FIG. 10 shows the % cumulative release based on PAMPA and PAMPA with mucus.

FIG. 11 is a UV spectrum relating to the quantification of glycans on the surface of NPs-MUCGIi with PAS reagent (example 5).

FIG. 12 shows the fluorescence spectra of concanavalin A lectin in the presence of increasing concentrations of NPs-MUCGli.

FIG. 13 regards: (A) the percentage of bacterial viability over time for the NPs-MUCGli, the NPs-MUCGli/cipro of the invention and ciprofloxacin compared to the standard, evaluated with respect to S. aureus; and (B) the percentage of bacterial viability over time for the NPs-MUCGIi, the NPs-MUCGli/cipro of the invention and ciprofloxacin compared to the standard, evaluated with respect to P. aeruginosa.

FIG. 14 shows the cellular internalisation and intracellular distribution of the NPs-MUCGli/FITC nanoparticles. The NPs-MUCGli/FITC nanoparticles were incubated at a concentration of 25 μg/mL with HaCaT cells for 5 hours. The cell membranes were stained with calcein.

FIG. 15 shows a UV-vis comparison of the nanoparticles described in Yan et al. (PGM Yan NPs), the nanoparticles obtained with the method of the application (NPs-MUCGli) and PGM.

FIG. 16 shows a comparison of TEM images: A) NPs-MUCGli obtained from porcine gastric mucin type III with the method described in the present patent application; B) NPs PGM Yan obtained from porcine gastric mucin type III with the glycerol/H₂O-based method described in the literature by Yan et al.

FIG. 17 shows the UV-visible (UV-vis) spectrum of the NPs-MUCGli obtained in example compared to the UV-vis spectrum of BSM alone.

FIG. 18 shows the TEM (transmission electron microscope) images of the NPs-MUCGli obtained in example 10.

FIG. 19 shows the characterisation, in LC-MS/MS, of the active ingredient pralatrexate (A, B, C).

FIG. 20 shows the characterisation, in LC-MS/MS, of the active ingredient remdesivir (A, B, C).

FIG. 21 shows the characterisation, in LC-MS/MS, of the active ingredient camostat (A, B, C).

FIG. 22 shows the characterisation, in LC-MS/MS, of the active ingredient RSV604 (A, B, C).

FIG. 23 shows the activity of the NPs-MUCGli and NPs-MUCGli/prala on Sars-Cov-2.

FIG. 24 shows the characterisation, in LC-MS/MS, of the active ingredient doxorubicin (A, B, C).

FIG. 25 shows the characterisation, in LC-MS/MS, of the active ingredient trametinib (A, B, C).

FIG. 26 describes the antiproliferative activity of the NPs-MUCGli and NPs-MUCGli/trame (A) and NPs-MUCGli/doxo (B) on the H358 cell line.

FIG. 27 shows the characterisation of the NPs-MUCGli/cy5.5: A) UV-vis spectra of the supernatants used to measure the effectiveness of encapsulation; B) FESEM image; C) Dynamic Light Scattering (DLS) analysis.

FIG. 28 shows results obtained from the experiment of optical imaging in vivo.

FIG. 29 shows the monitoring of the weight of treated and untreated animals.

FIG. 30 shows the characterisation, in LC-MS/MS, of the active ingredient ceftazidime (A, B, C).

FIG. 31 shows the characterisation of the active ingredient azithromycin (A, B, C).

FIG. 32 shows the characterisation, in LC-MS/MS, of the active ingredient dexamethasone (A, B, C).

FIG. 33 show the characterisation, in LC-MS/MS, of the active ingredient baricitinib (A, B, C).

FIG. 34 shows the fluorescence spectra of the supernatants used to measure the effectiveness of encapsulation for albumin bioconjugated with FITC.

FIG. 35 shows a representative bar graph (for three independent experiments), which shows the viability of HeLa cells in the presence of NPs-MUCGli at different concentrations (n=8 replications/experiment).

FIG. 36 describes the results of cytokine release in the presence of NPs-MUCGli on a human macrophage cell line.

FIG. 37 shows the results obtained for blood coagulation parameters after the addition of NPs-MUCGli.

FIG. 38 is a schematic illustration of the absorption and loss of mass when the NPs-MUCGIi are adsorbed and desorbed from the surface of the QCM-D sensor.

FIG. 39 shows the quantification of sialic acid present in PGM and in the NPs-MUCGli.

FIG. 40 shows the fluorescence spectra of the supernatants used to measure the effectiveness of encapsulation for PNA bioconjugated with FITC.

### DETAILED DESCRIPTION OF PREFERRED EMBODIMENTS OF THE INVENTION

The subject matter of the present invention relates to covalently cross-linked glycosylated mucin nanoparticles, wherein the mucin oligosaccharide chains, responsible for glycosylation, are arranged on the surface of the nanoparticles.

The composition of the oligosaccharide part of the mucins also includes sialic acid, where said term indicates the N- and O-substituted derivatives of neuraminic acid, i.e. a monosaccharide and 9 carbon atoms. N-acetylneuraminic acid is preferred in particular.

The covalently cross-linked glycosylated mucin nanoparticles according to the present invention advantageously have glycans on the surface, as demonstrated in the experimental part (PAS test—example 5), though in a smaller amount than the starting mucin.

The glycan chains present on the surface of the covalently cross-linked glycosylated mucin nanoparticles of the present invention comprise N-acetylgalactosamine, N-acetylglucosamine, fucose, galactose and/or sialic acid.

The subject matter of the present invention thus relates to covalently cross-linked glycosylated mucin nanoparticles, on their own or loaded with at least one compound selected from an active ingredient, a marker and a biomolecule, wherein the surface oligosaccharide chains comprise N-acetylgalactosamine, N-acetylglucosamine, fucose, galactose and/or sialic acid.

Glycosylation, i.e. the addition of carbohydrates or sugars, is widespread in nature as a means of functionalising molecules for cell recognition and signalling, and takes place thanks also to the interaction between oligosaccharides and lectins. In the case of the covalently cross-linked glycosylated mucin nanoparticles of the present invention, the oligosaccharide chains are naturally present on the mucin and thus need not be added separately through a process of synthesis. The oligosaccharide chains are arranged on the surface of the nanoparticles and are thus available for interaction with the lectins present, for example, on bacteria and for interaction with viruses, thanks to the sialic acids and lectins present in the oligonucleotide chains themselves, which viruses can bind with, in particular influenza viruses, parainfluenza viruses, mumps virus, coronaviruses, noroviruses, rotaviruses and DNA tumour viruses.

Their ability to incorporate a good amount of active ingredients and deliver them to bacterial lectins and viruses makes the nanoparticles according to the present invention suitable carriers for antibacterial and/or antiviral active ingredients that can be delivered directly to the site of bacterial and/or viral infection and released there. This makes it possible to render the antibacterial and/or antiviral active ingredients more available precisely at the specific site of action and thereby contribute to overcoming bacterial and/or viral resistance.

Furthermore, lectins are implicated in the formation of bacterial biofilm, a complex aggregation of microorganisms distinguished by the secretion of a protective adhesive matrix that renders bacteria even more resistant to the attack of antibiotics.

The covalently cross-linked glycosylated mucin nanoparticles of the invention can thus interfere with the lectins that form biofilms and release the antibiotic in a targeted fashion, thus contributing in this way as well to overcoming bacterial resistance.

Furthermore, the covalently cross-linked glycosylated mucin nanoparticles of the invention have a mucoadhesive activity, which allows the bacterial aggregates to be reached so that the antibiotic can be released at the site of action and inhibit the formation of a biofilm.

Therefore, the covalently cross-linked glycosylated mucin nanoparticles of the invention, which are biocompatible and do not have immunogenic activity, can be used as carriers for the delivery of active ingredients, markers and/or biomolecules, as they are able to reach the target sites thanks to the surface glycosylation, as demonstrated in the experimental part (interaction with concanavalin A lectin).

The covalently cross-linked glycosylated mucin nanoparticles according to the invention, comprising at least one antibacterial active ingredient, are used in the treatment of pathologies involving at least one bacterial strain resistant to at least one defined antibiotic. This preferably takes place in patients undergoing a simultaneous or sequential treatment with a given antibiotic said bacterial strain shows resistance to.

The covalently cross-linked glycosylated mucin nanoparticles according to the invention, comprising at least one antibacterial active ingredient, are used in the treatment of pathologies involving a wild-type bacterial strain that does not have acquired resistance to any known antibiotic. This preferably takes place in patients undergoing a simultaneous or sequential treatment with a “prescribed antibiotic” said bacterial strain does not show resistance to.

The covalently cross-linked mucin nanoparticles of the present invention are moreover usable for treating pathologies in which there is an overproduction of mucus, such as cystic fibrosis, chronic obstructive pulmonary disease and bronchiectasis. Advantageously, the covalently cross-linked glycosylated mucin nanoparticles according to the present invention act as carriers and, being mucoadhesive, they favour the release of the at least one active ingredient, marker and/or biomolecule delivered to the desired site. Said nanoparticles, in fact, dissolve in mucus, releasing the at least one compound selected from an active ingredient, a marker and a biomolecule directly at the target site.

The covalently cross-linked glycosylated mucin nanoparticles of the invention are thus capable of releasing the active ingredient directly in contact with viruses also in the presence of a thick layer of mucus, which can hinder the release of active ingredients at viral infection sites.

This aspect is therefore particularly advantageous in the event of viral infections that lead to an excessive production of mucus in the respiratory tract, such as, for example, the infection caused by the SARS-CoV-2 virus (Severe Acute Respiratory Syndrome-CoronaVirus-2—name according to the International Committee on Taxonomy of Viruses (ICTV)).

The covalently cross-linked mucin nanoparticles according to the present invention comprising markers can be advantageously used in molecular imaging for the visualisation of proteins present on the surface of neurons.

The subject matter of the invention thus relates to the covalently cross-linked glycosylated mucin nanoparticles as defined above as a medicament, preferably as antivirals, or for use as carriers of at least one compound selected from an active ingredient, a marker and a biomolecule.

The covalently cross-linked glycosylated mucin nanoparticles of the invention represent multifunctional glycomimetic nanosystems (glycomimetic carriers) capable of incorporating good amounts of active ingredients, markers and/or biomolecules: the efficiency of entrapment of these substances is comprised between 70% and 95%.

The complete release of the active ingredients, markers and/or biomolecules by the covalently cross-linked glycosylated mucin nanoparticles of the invention takes place after 24 hours (data calculated in vitro by dialysis and centrifugation), since there is no covalent bond that needs to be broken between the loaded active ingredient and the nanoparticles of the invention.

The covalently cross-linked glycosylated mucin nanoparticles of the invention have an average particle diameter comprised between 100 and 400 nanometres (nm), preferably between 150 and 300 nm and even more preferably about 250 nm. The average particle diameter of the nanoparticles of the invention was measured by means of a transmission electron microscope (TEM) and also confirmed with DLS (Dynamic Light Scattering) methods.

The subject matter of the invention thus relates to covalently cross-linked glycosylated mucin nanoparticles wherein the mucin oligosaccharide chains, responsible for glycosylation, are arranged on the surface of the nanoparticles, comprising at least one compound selected from an active ingredient, a marker and a biomolecule.

The term “comprising” means that the nanoparticles encapsulate at least one compound selected from an active ingredient, a marker and a biomolecule.

Active ingredients of particular interesse that can be carried by the covalently cross-linked glycosylated mucin nanoparticles of the invention are antibiotics. Particularly preferred antibiotics are selected from antibiotics belonging to the class of aminoglycosides, cephalosporins, quinolones, lincosamides, macrolides, nitroimidazoles, penicillins, sulphonamides, tetracyclines and/or peptides. Among quinolone antibiotics, ciprofloxacin is particularly preferred; among cephalosporin antibiotics, ceftazidime is particularly preferred; and among macrolide antibiotics, azithromycin is particularly preferred.

Other active ingredients of particular interest that can be carried by the covalently cross-linked glycosylated mucin nanoparticles of the invention are antiviral active ingredients.

Antiviral active ingredients of particular interest are selected from active ingredients that are active against influenza viruses, herpes viruses, hepatic viruses, HIV and/or viruses of the Poxviridae family. Active ingredients that are active against the SARS-CoV-2 (commonly called Covid-19) virus are also of particular interest: particularly preferred is the active ingredient remdesivir or mixtures of antiviral active ingredients, such as, for example, lopinavir/ritonavir, darunavir/ritonavir and darunavir and cobicistat. Further antiviral active ingredients against the SARS-CoV-2 virus of particular interest, which can be carried by the covalently cross-linked glycosylated mucin nanoparticles of the invention, are the active ingredients camostat and pralatrexate. RSV 604 can also be carried for the respiratory syncytial virus.

Further active ingredients of particular interest that can be carried by the covalently cross-linked glycosylated mucin nanoparticles of the invention are antitumoural active ingredients. Antitumoural active ingredients of particular interest are selected from alkylating agents, antimetabolites, antitumoural antibiotics, topoisomerase inhibitors, differentiated agents and/or the active ingredients that stimulate the immune system.

Particularly preferred antitumoural active ingredients are doxorubicin and trametinib.

Further active ingredients of particular interest that can be carried by the covalently cross-linked glycosylated mucin nanoparticles of the invention are steroidal anti-inflammatory active ingredients, such as, for example, dexamethasone, and non-steroidal anti-inflammatory active ingredients, such as, for example, baricitinib.

Markers of particular interest that can be carried by the covalently cross-linked glycosylated mucin nanoparticles of the invention are fluorophores. Particularly preferred fluorophores are fluorescein isothiocyanate, rose bengal and/or near-infrared fluorophores, such as cyanines, and in particular cyanine 5.5.

Biomolecules of particular interest that can be carried by the covalently cross-linked glycosylated mucin nanoparticles of the invention are nucleic acids, peptides, lipids and/or growth factors.

It should also be stressed that the sugar chains forming the surface glycosylation of the covalently cross-linked mucin nanoparticles of the invention can also be modified so as to insert new types of sugars in said sugar chains. For example, the surface glycans of the nanoparticles of the invention can also be functionalised with α-L-fucose and/or one or more compounds of the lectin class, such as, for example, concanavalin A, haemagglutinin (HA), neuraminidase (NA), lectin A (LecA) and/or lectin B (LecB), and thus have application for use in mucosal vaccinations and in the treatment of the viral infections. The inclusion of α-L-fucose enables the recognition of bacteria (e.g. Helicobacter pylori) or several viruses and thus the release of the antibacterial active or antiviral ingredient directly at the target (Steven L. Taylor et al. Trends in Microbiology, February 2018, Vol. 26, No. 2 https://doi.org/10.1016/j.tim.2017.09.011).

Therefore, the subject matter of the present invention relates to covalently cross-linked glycosylated mucin nanoparticles, wherein the mucin oligosaccharide chains, responsible for the glycosylation, are arranged on the surface of the nanoparticles, optionally comprising at least one compound selected from an active ingredient, a marker and a biomolecule, wherein the oligosaccharides of the mucin are further functionalised, for example with α-L-fucose and/or one or more compounds of the lectin class, such as, for example, concanavalin A, haemagglutinin (HA), neuraminidase (NA), lectin A (LecA) and/or lectin B (LecB).

Glycosylated nanosystems that envisage a process of surface functionalisation of the nanoparticles with carbohydrates are described in the literature. Said glycosylation process is a complex chemical process that requires various steps of synthesis, making the transfer of the process of synthesis onto an industrial scale complex and/or not cost-effective.

The covalently cross-linked glycosylated mucin nanoparticles of the present invention, by contrast, are obtained according to a “one-pot” preparation method that makes it possible to obtain said mucin nanoparticles, directly functionalised, on the outer surface, with the oligosaccharides present in the starting mucin, optionally comprising at least one compound selected from an active ingredient, a marker and a biomolecule. The starting mucin used to prepare the covalently cross-linked glycosylated mucin nanoparticles of the present invention, on their own or comprising at least one compound selected from an active ingredient, a marker and a biomolecule, is not functionalised and is not marked.

Therefore, the subject matter of the present invention further relates to a “one-pot” method for preparing covalently cross-linked glycosylated mucin nanoparticles, which comprises the following steps:
a) obtaining a solution, preferably a saline solution, of mucin;
b) adjusting the pH of the solution obtained in the preceding step to a pH comprised from 7.5 to 9.5, preferably from 8 to 9, and even more preferably to pH 8.5;
c) desolvating the mucin by adding an alcoholic solvent, preferably ethanol, to the solution directly obtained in step b);
d) adding a cross-linker, preferably glutaraldehyde, to the solution directly obtained in step c).

According to a preferred aspect the glutaraldehyde is added at a speed of one drop per second. The reaction takes place at room temperature.

After the addition of the cross-linker, the solution is kept under stirring for a time comprised between 4 and 48 h, preferably for 12-24 h.

The nanoparticles thus obtained in solution can be easily purified; they are preferably purified by centrifugation.

The above-described method can thus comprise a further step of purifying the mucin nanoparticles obtained, preferably a step of purification by centrifugation.

The purified nanoparticles of the invention can be lyophilised to obtain the powder form.

The above-described method can thus comprise a further step of lyophilising the purified mucin nanoparticles.

In a preferred embodiment, the method is based on commercial mucins deriving from pig stomach, i.e. porcine gastric mucin (PGM) is used, or the method is based on bovine submaxillary mucin (BSM). According to a further preferred aspect, the porcine gastric mucin is porcine gastric mucin type III.

According to a preferred aspect, the cross-linker used in the method of the invention is glutaraldehyde, as it is a nontoxic compound; however, other covalent cross-linkers with low toxicity can likewise be used.

According to another preferred aspect, the mucin solution of step a) is a solution of NaCl.

According to a further preferred aspect, the alcoholic solvent, preferably ethanol, is added dropwise.

The above-described method can thus comprise a step in which the alcoholic solvent, preferably ethanol, is added dropwise.

Advantageously, the method according to the present invention enables covalently cross-linked glycosylated mucin nanoparticles to be obtained directly, without having to rely on subsequent functionalisation of the nanoparticles with oligosaccharides.

Once the glycosylated mucin nanoparticles have been obtained, it is possible to further functionalise the outer oligosaccharide chains, for example with α-L-fucose and/or one or more compounds of the lectin class, such as, for example, concanavalin A, haemagglutinin (HA), neuraminidase (NA), lectin A (LecA) and/or lectin B (LecB).

Therefore, the subject matter of the invention also relates to covalently cross-linked glycosylated mucin nanoparticles obtainable with the above-described “one-pot” preparation method.

According to a further preferred aspect, the “one-pot” method of the invention for preparing mucin nanoparticles can be used to produce covalently cross-linked glycosylated mucin nanoparticles comprising at least one compound selected from an active ingredient, a marker and a biomolecule.

Said “one-pot” method for preparing covalently cross-linked glycosylated mucin nanoparticles comprising at least one compound selected from an active ingredient, a marker and a biomolecule, in addition to steps a) to d) described above, further comprises a step a’) between step a) and step b):
a′) adding at least one compound selected from an active ingredient, a marker and a biomolecule to the solution obtained in step a).

Therefore, the subject matter of the invention further relates to a “one-pot” method for preparing covalently cross-linked glycosylated mucin nanoparticles comprising at least one compound selected from an active ingredient, a marker and a biomolecule, which comprises the following steps:
a) obtaining a solution, preferably a saline solution, of mucin;
a′) adding at least one compound selected from an active ingredient, a marker and a biomolecule to the solution obtained in step a);
b) adjusting the pH of the solution obtained in the preceding step to a pH comprised from 7.5 to 9.5, preferably from 8 to 9, and even more preferably to pH 8.5;
c) desolvating the mucin by adding an alcoholic solvent, preferably ethanol, to the solution directly obtained in step b);
d) adding a cross-linker, preferably glutaraldehyde, to the solution directly obtained in step c).

According to a preferred aspect, the glutaraldehyde is added at a speed of one drop per second. The reaction takes place at room temperature.

After the addition of the cross-linker, the solution is kept under stirring for a time comprised between 4 and 48 h, preferably for 12-24 h.

The nanoparticles thus obtained in solution can be easily purified; they are preferably purified by centrifugation.

The method above described can thus comprise a further step of purifying the mucin nanoparticles obtained, preferably a step of purification by centrifugation.

The purified nanoparticles of the invention can be lyophilised to obtain the powder form.

The method above described can thus comprise a further step of lyophilising the purified mucin nanoparticles.

In a preferred embodiment, the method is based on commercial mucins deriving from pig stomach, i.e. porcine gastric mucin (PGM) is used, or the method is based on bovine submaxillary mucin (BSM). According to an aspect further preferred, the porcine gastric mucin is porcine gastric mucin type III.

According to a preferred aspect, the cross-linker used in the method of the invention is glutaraldehyde, as it is a nontoxic compound; however, other covalent cross-linkers with low toxicity can likewise be used.

According to a preferred aspect, the mucin solution of step a) is a solution of NaCl.

According to a further preferred aspect, the alcoholic solvent, preferably ethanol, is added dropwise.

The method above described can thus comprise a step in which the alcoholic solvent, preferably ethanol, is added dropwise.

Advantageously, the method according to the present invention enables covalently cross-linked glycosylated mucin nanoparticles, comprising at least one compound selected from an active ingredient, a marker and a biomolecule, to be obtained directly, without having to rely on subsequent functionalisation with oligosaccharides.

Once the covalently cross-linked glycosylated mucin nanoparticles have been obtained, it is possible to further functionalise the outer oligosaccharide chains, for example with α-L-fucose and/or one or more compounds of the lectin class, such as, for example, concanavalin A, haemagglutinin (HA), neuraminidase (NA), lectin A (LecA) and/or lectin B (LecB).

Therefore, the subject matter of the invention relates to glycosylated mucin nanoparticles comprising at least one compound selected from an active ingredient, a marker and a biomolecule, obtained with the above-described “one-pot” preparation method.

Pharmaceutical formulations containing the covalently cross-linked glycosylated mucin nanoparticles, optionally comprising at least one compound selected from an active ingredient, a marker and a biomolecule described herein can be prepared using a physiologically acceptable excipient which is considered safe and effective and can be administered to an individual without causing undesirable biological effects or undesirable interactions.

The covalently cross-linked glycosylated mucin nanoparticles of the present invention can be formulated for oral, inhalational and/or parenteral administration. They can in fact be purified and lyophilised and this makes it possible to obtain sterile solutions thereof, which may be used, for example, to prepare formulations for aerosol administration and injection. When used, for example, for mucosal vaccination, the covalently cross-linked glycosylated mucin nanoparticles of the invention can be formulated in oral form.

The invention is illustrated below by means of experimental examples, which are not to be considered limiting for the object of the invention.

### EXAMPLES

### Example 1

### Preparation of Covalently Cross-Linked Glycosylated Porcine Gastric Mucin Nanoparticles (NPs-MUCGIi)

50 mg of commercial porcine gastric mucin (PGM) type III (Sigma Aldrich Partially purified powder, Cas Number 84082-64-4) were weighed. Mucin type III is a partially purified porcine gastric mucin powder prepared according to the method described in Glenister, D. A. and Salamon, K. Microbial Biology in Health & Disease 1, 31, (1988). Then 2 ml of 10 mM NaCl were added. The solution was left under stirring for 4 hours; the pH was brought to 8.5 with 0.1 mM NaOH. 8 ml of ethanol 1 gtt/sec were added. Then 90 μl of 8% glutaraldehyde in milli-q water (cross-linking agent) were added and the solution was left under stirring for 24 hours.

The nanoparticles obtained in solution were purified by centrifugation: they were transferred into a Falcon tube and 5 centrifugation cycles were carried out: 1) 1000 rpm×5 min; 2) 2000 rpm×5 min; 3) 4000 rpm×5 min; 4) 4000 rpm×15 min; 5) 4000 rpm×15 min.

The supernatant was removed at every step and replaced with 1 ml of fresh milli-Q water after every centrifugation cycle.

A solid was obtained from the centrifugation and resolubilised in aqueous solution.

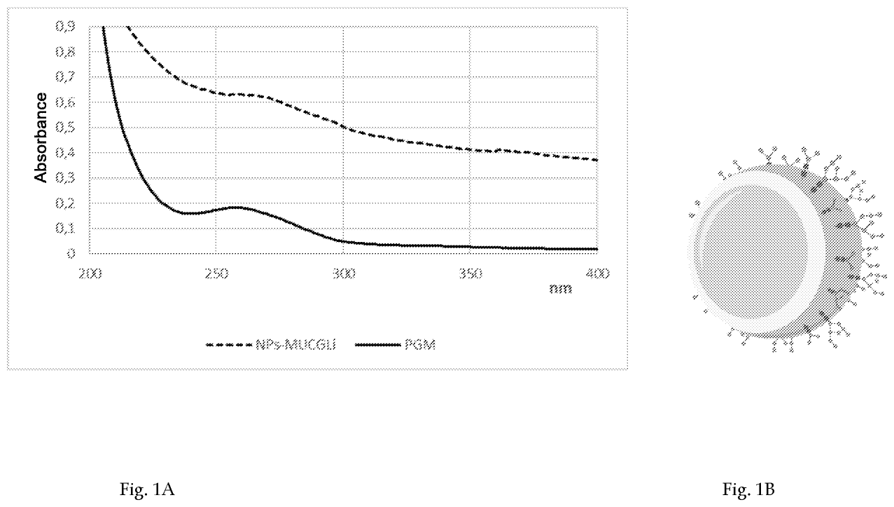

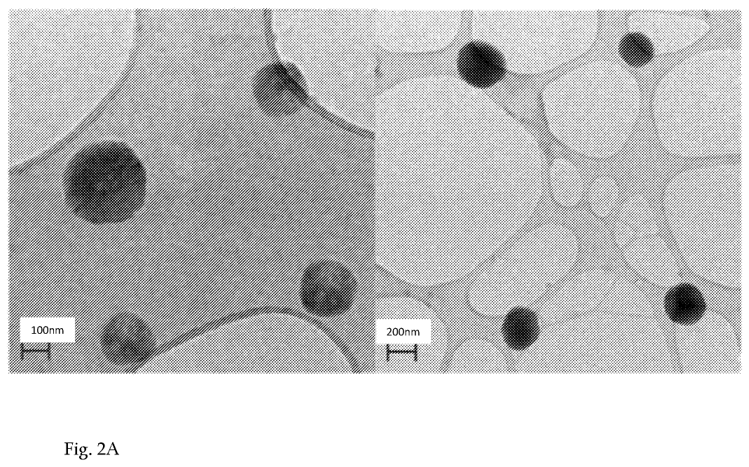

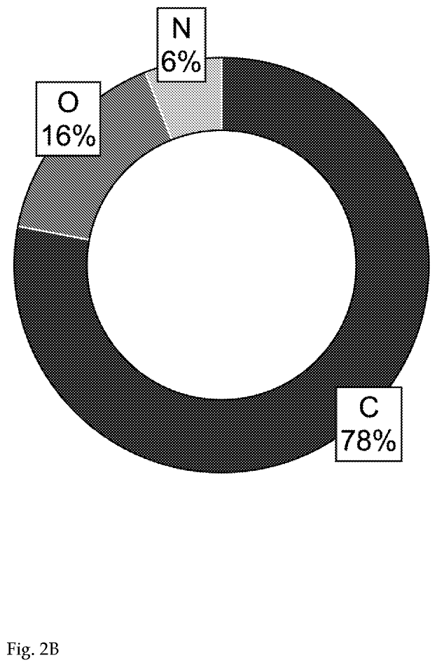

The NPs-MUCGli were lyophilised to obtain the powder form: the sample was divided into three 2 mL aliquots in three different Eppendorf® test tubes and placed in liquid nitrogen until completely frozen. The flask was then connected to a tabletop freeze dryer (HETO LyoLab 3000) equipped with a refrigerator, vacuum centrifuge and vacuum pump to maintain the temperature a −56° C. for a period of 8 hours. The NPs-MUCGli were resuspended in 2 ml of milli-q water and compared with the original sample by means of a UV-Vis study (FIG. 1A) and TEM and it was observed that the nanoparticles maintain their characteristics (FIG. 2A).

Characterisation of NPs-MUCGli Nanoparticles

After the synthesis of the NPs-MUCGli, a UV-Vis study was conducted: 100 μL of NPs-MUCGIi were diluted to 1 ml with milli-q water (900 μL). The spectrum was measured at 25° C. in the 200-400 nm interval (FIG. 1A).

The synthesised nanoparticles were characterised by TEM (model JEOL 3010-UHR) (FIG. 2A).

Via an EDS X-ray qualitative microanalysis it was also determined that C, N and O are the only elements making up the NPs-MUCGli (FIG. 2B). The size of the NPs-MUCGli is comprised between 200 and 300 nm.

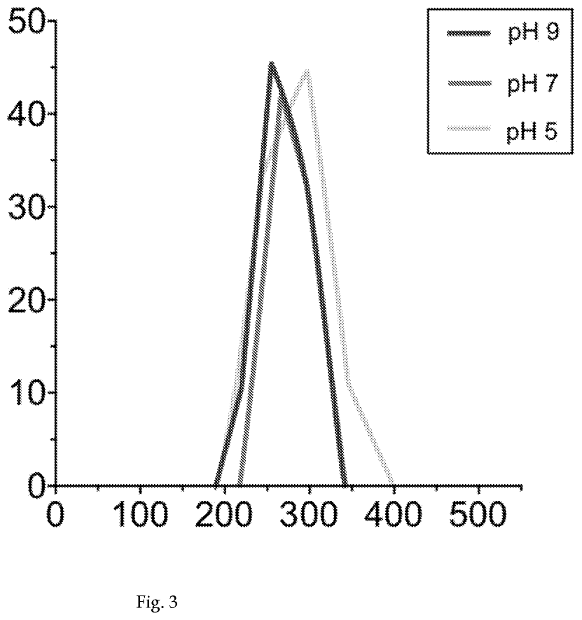

A further characterisation was performed with the Dynamic Light Scattering (DLS) technique and the sizes obtained by TEM were confirmed. The experiment was conducted at different pH values (FIG. 3).

### Example 2

Preparation of Covalently Cross-Linked Glycosylated Porcine Gastric Mucin Nanoparticles Loaded with Ciprofloxacin (NPs-MUCGli/Cipro).

The NPs-MUCGli/cipro were synthesised with the same desolvation method as used for the NPs-MUCGli of example 1. For this preparation, 50 mg of PGM type III were solubilised in 2.0 ml of a 10 mM NaCl solution. 3 mg of ciprofloxacin were added to the resulting opalescent solution and incubated for 4 hours. The resulting solution was brought to pH 8.5 with a 0.1 mM NaOH solution. Then 8.0 ml of ethanol were continuously added at room temperature and under vigorous stirring. It was possible to observe the beginning of nanoparticle formation by precipitation. The ethanol flow rate was set at 1 gtt/sec. After the desolvation process, 90 μl of 8% glutaraldehyde (in milli-q water) were added to induce the cross-linking of the particles, which was completed after the suspension had been left under stirring for 24 hours.

The resulting NPs-MUCGli/cipro were purified as described in example 1.

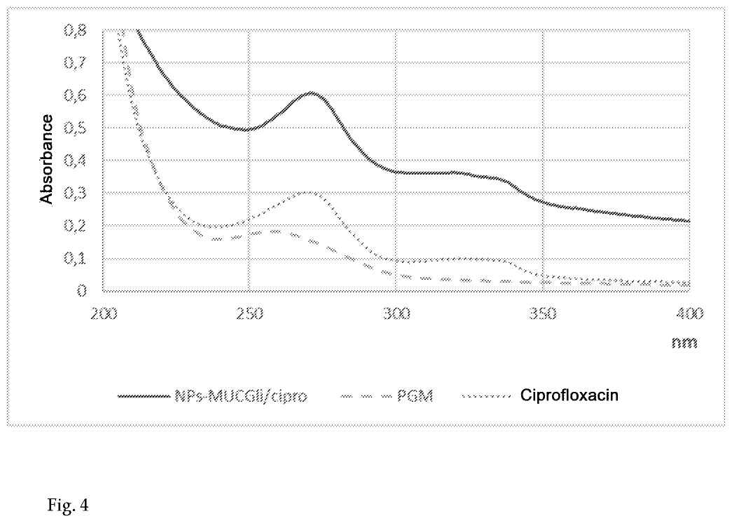

The NPs-MUCGli/cipro maintained their properties for one week of storage at 4° C. The NPs-MUCGli/cipro were lyophilised to obtain the powder form with the same technique as described in example 1. The NPs-MUCGli/cipro were then resuspended in 3 ml of milli-q water and compared with the original sample by means of a UV-Vis study (FIG. 4).

Encapsulation Efficiency of NPs-MUCGli/Cipro.

In order to quantify the exact amount of ciprofloxacin encapsulated in the NPs-MUCGli/cipro, a UV study was conducted on the supernatants collected during the step of purifying the NPs-MUCGli/cipro. 100 μL of supernatant were withdrawn and diluted to 1 ml with milli-q water (900 μL); the spectrum was again measured in the 200-400 nm interval at maximum absorbance at 323 nm.

The concentration of ciprofloxacin (μg/mL) was determined by replacing this value in the ciprofloxacin calibration curve.

Therefore, in 1 ml the mass of ciprofloxacin (μg) was the same as its concentration ([ciprofloxacin] (μg/ml)×1 ml).

The mass of ciprofloxacin in the solution of supernatants was then calculated: the mass value was multiplied by the volume of supernatants collected and divided by 100 μL. This amount was removed from the drug initially added (3 mg) and the mass of encapsulated ciprofloxacin was determined to be 700 ug/mL.

Characterisation of NPs-MUCGli/Cipro Nanoparticles

The NPs-MUCGli/cipro were characterised via UV-vis (FIG. 4).

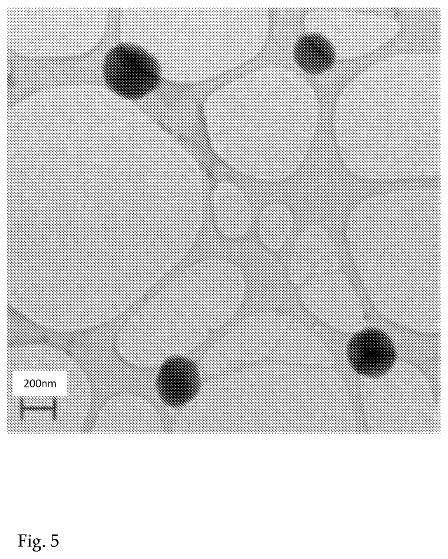

The synthesised NPs-MUCGli/cipro were characterised by TEM (model JEOL 3010-UHR) (FIG. 5). The sizes showed to be similar to those of the NPs-MUCGli, i.e. the particle diameter was 200 nm.

Release of Ciprofloxacin by NPs-MUCGli/Cipro

The NPs-MUCGli/cipro were synthesised as described above. The concentrations of ciprofloxacin and mucin were then quantified as described above. In order to quantify the mucin, 100 μL of a sample were collected and diluted to 1 ml with milli-q water (900 μL); the spectrum was measured at 25° C. in the 200-400 nm interval. The absorbance was measured at 256 nm and substituted into the calibration curve.

In order to determine the concentration of ciprofloxacin ([ciprofloxacin]), by contrast, the supernatants were collected during purification; 100 μL were withdrawn and diluted to 1 ml with milli-q water (900 μL); the spectrum was measured at 25° C. in the 200-400 nm interval.

The absorbance was measured at 323 nm and substituted into the calibration curve.

The following concentrations were determined:
[mucin]=369 μg/mL
[ciprofloxacin]=76.8 μg/mL

Therefore, a mucin and ciprofloxacin solution was prepared in order to have the same concentrations as in the NPs-MUCGli/cipro. In order to prepare a solution in which the concentration of mucin was 369 μg/mL and the concentration of ciprofloxacin was 76.8 μg/mL, 1.5 mg of PGM type III were weighed on an analytical balance and diluted to a final volume of 4 mL with 10 mM PBS. Then 0.3 mg of ciprofloxacin were added and the solution was mixed with a vortex mixer.

The experiment was conducted using a Spectra/Por 1® membrane with the following characteristics:

| | | |
|---|---|---|
| Molecular Weight Cut-Off: | 6-8 | kD |
| Flat Width: | 23 | mm |
| Diameter: | 14.6 | mm |
| Volume/Length: | 1.7 | mL/cm |

Every membrane was filled with 1 mL of solution of NPs-MUCGli/cipro or mucin and ciprofloxacin solution, and closed with dialysis clips.

Every 5, 10, 15, 30 minutes, 1 hour, 2 hours, 4 hours, 6 hours and 24 hours, 200 μL of receptor medium were withdrawn and replaced with the same amount of fresh PBS. The samples were transferred into an Eppendorf® and diluted to 1 mL (800 μL) with 10 mM PBS and a UV-Vis study was conducted in the 200-400 nm interval. In order to quantify the concentration of ciprofloxacin, the absorbance was measured at 323 nm and substituted into the calibration curve. After the concentration was calculated (μg/mL), the mass of ciprofloxacin was determined. Table 1 show the quantification of the NPs-MUCGli/cipro, wherein the abbreviation “abs” stands for absorbance.

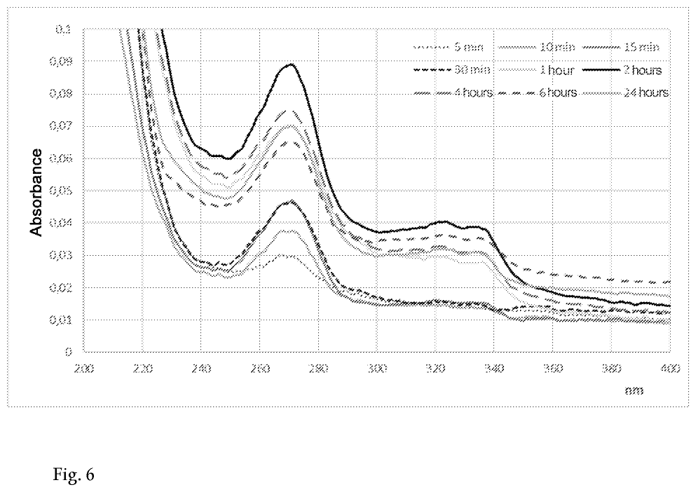

The UV-vis spectra obtained during the monitoring of the release of ciprofloxacin are shown in FIG. 6.

TABLE 1

| | | | Concentration | Mass in 1 mL | Total Mass |
|---|---|---|---|---|---|
| Time | | Abs | (μg/mL) | (μg) | (μg) |
| 5 | min | 0.0145 | 0.221 | 9.94 | 70.72 |
| 10 | min | 0.0148 | 0.221 | 9.96 | 141.55 |
| 15 | min | 0.0158 | 0.222 | 10.01 | 212.70 |
| 30 | min | 0.0157 | 0.222 | 9.99 | 283.79 |
| 1 | h | 0.0297 | 0.236 | 10.63 | 359.36 |
| 2 | h | 0.0405 | 0.247 | 11.12 | 438.40 |
| 4 | h | 0.0327 | 0.239 | 10.76 | 514.93 |
| 6 | h | 0.0362 | 0.243 | 10.92 | 592.59 |
| 24 | h | 0.032 | 0.239 | 10.73 | 668.9 |

### Example 3

Preparation of Covalently Cross-Linked Glycosylated Porcine Gastric Mucin Nanoparticles Loaded with Fluorescein Isothiocyanate (NPs-MUCGli/FITC).

The NPs-MUCGli/FITC were synthesised with the same desolvation method as the NPs-MUCGIi of example 1.

50 mg of PGM type III were solubilised in 2.0 ml of a 10 mM NaCl solution. 3 mg of FITC were added to the resulting opalescent solution and incubated for 4 hours. The resulting solution was brought to pH 8.5 with a 0.1 mM NaOH solution. Then 8.0 ml of ethanol were continuously added at room temperature and under vigorous stirring. It was possible to observe the beginning of nanoparticle formation. The amount of ethanol was set at 1 gtt/sec. After the desolvation process, 90 μl of 8% glutaraldehyde (in milli-q water) were added to induce the cross-linking of the particles, which was completed after the suspension had been left under stirring for 24 hours.

The nanoparticles were purified with the method described in the previous example 1.

Characterisation of NPs-MUCGli/FITC

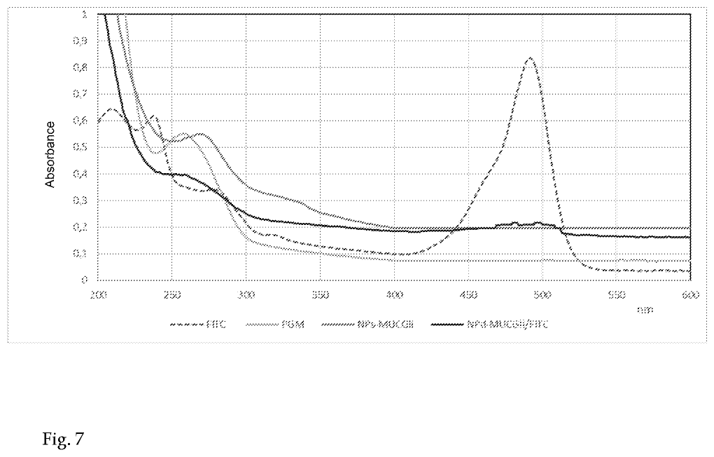

The NPs-MUCGli/FITC were initially characterised by UV-Vis spectroscopy. This spectrum was compared with that of PGM, FITC, NPs-MUCGli and NPs-MUCGli/FITC (FIG. 7).

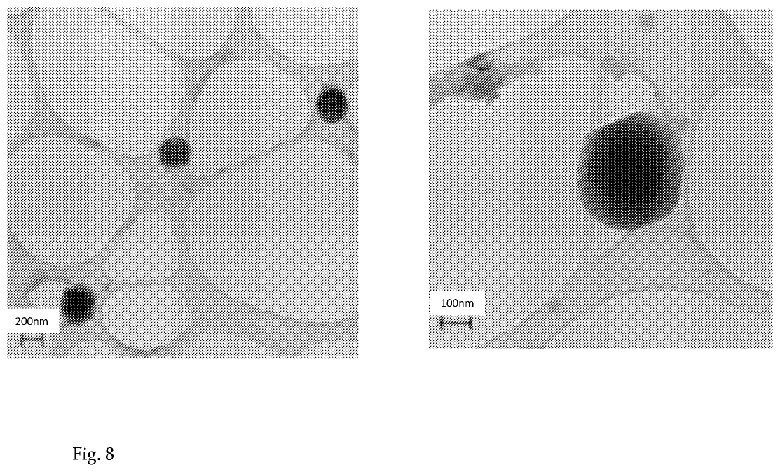

Thanks to transmission electron microscopy we were able to monitor the morphology of the NPs-MUCGli/FITC: the TEM images of the sample show that the NPs-MUCGli/FITC are spherical (FIG. 8). From the TEM images we established that the mean size of the NPs-MUCGli/FITC is 314.2±43.21 nm.

### Example 4

Permeability studies on NPs-MUCGli/cipro (PAMPA) in the absence and presence of a mucus model (as described in Pacheco, D. P., Butnarasu, C. S., Briatico Vangosa, F., Pastorino, L., Visai, L., Visentin, S., Petrini, P. “Disassembling the complexity of mucus barriers to develop a fast screening tool for early drug discovery” (2019) Journal of Materials Chemistry B, 7 (32), pp. 4940-4952).

The experiment was conducted on a Corning® PAMPA pre-coated 96-well plate system. The porosity of the artificial membrane is 0.45 μm and the internal diameter is 6.4 mm. The diffusion of the drug through the PAMPA membrane was evaluated both in the absence and in the presence of mucus; the diffusion of propranolol was also studied as a standard, since it is classified as a high-permeability drug.

Then, before the drug release test, a permeability study was conducted using the traditional formula (equation 1—eq. 1):

$$Pe = \frac{- ln [ 1 - \frac{CA ( t )}{C equilibrium}}{A ( \frac{1}{VD} + \frac{1}{V A} ) t} \qquad Eq . 1$$

where Pe is the effective permeability in units of cm/s, A=effective area of the filter, V_{D}=volume of the donor well (200 μL), V_{A}=volume of the acceptor well (300 μL), t=incubation time (i) of, C_{A} (t)=concentration of the compound in the acceptor well at time t, C_{D} (t)=concentration of the compound in the donor well at time t and

Cequilibrium=[CD(t)VD+CA(t)VA/(VD+VA) Eq.2

First of all, we prepared 200 μM of 1% v/v DMSO solutions of:
propranolol—200 μM
A 2 mM stock solution was prepared by solubilising 2.4 mg of propranolol in 40 μL of DMSO, diluted to a final volume of 4 mL with 10 mM of PBS (3960 μL). Then 400 μL of the stock solution were diluted to 4 ml with PBS 10 mM (3600 μL).
ciprofloxacin—200 μM
A 2 mM stock solution was prepared by solubilising 2.7 mg of ciprofloxacin in 40 μL of DMSO, diluted to a final volume of 4 ml with 10 mM of PBS (3960 μL). Then 400 μL of the stock solution were diluted to 4 ml with 10 mM PBS (3600 μL).

The donor compartment was filled with 200 μL of drug solution whereas the acceptor compartment was filled with 300 μL of 10 mM PBS. After 5 hours, the solutions were collected from the donor and acceptor compartments and analysed by fluorescence with a Fluorolog Jobin Yvon fluorometer.

The emission of propranolol was measured at 350 nm (A emission) after excitation at 289 nm (λ excitation); the spectrum was evaluated between 310 nm and 500 nm, with slits 3-4.

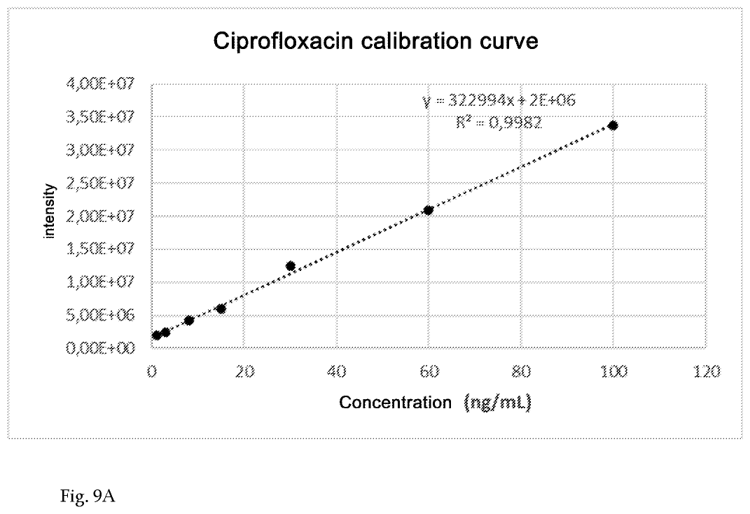

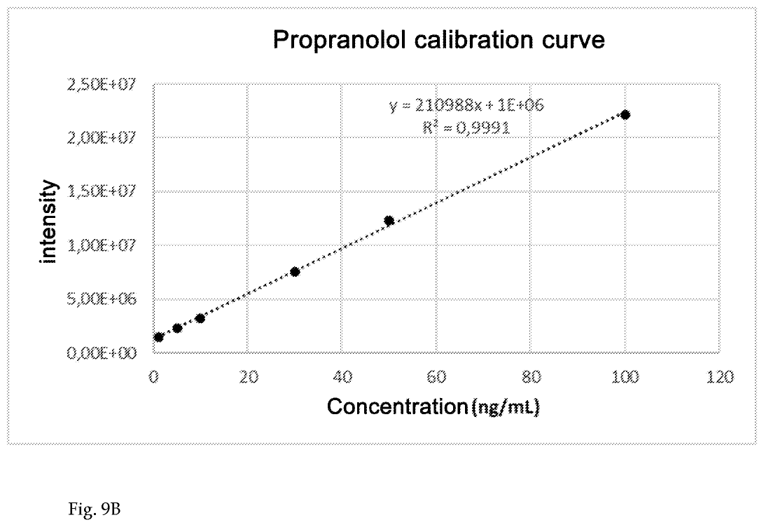

The emission of ciprofloxacin was measured at 418 nm (A emission) after excitation at 272 nm (λ excitation); the spectrum was evaluated between 290 nm and 500 nm, with slits 3-4 (FIG. 9).

The intensities were substituted into the calibration curves (FIG. 9) to determine the concentrations of ciprofloxacin and propranolol (ng/mL).

Table 2 shows the data related to the conditions used to obtain the permeability data.

TABLE 2
Concentrations of propranolol

| | Vd (mL) | Ca (t) (M) | Va (mL) | C0 (M) | A (cm2) | t (s) | Ceq (M) |
|---|---|---|---|---|---|---|---|
| 6.60E−06 | 0.2 | 2.01E−06 | 0.3 | 2.00E−04 | 0.3 | 18000 | 3.85E−06 |

Concentrations of ciprofloxacin

| Cd(t) (M) | Vd (mL) | Ca (t) (M) | Va (mL) | C0 (M) | A (cm2) | t (s) | Ceq (M) |
|---|---|---|---|---|---|---|---|
| 7.74E−05 | 0.2 | 5.70E−06 | 0.3 | 2.00E−04 | 0.3 | 18000 | 3.44E−05 |

The encapsulated mass was 782 μg (0.782 mg); considering the total volume (10 mL), the concentration of ciprofloxacin in the nanoparticles was determined to be 78.2 μg/mL (0.0782 mg/mL).

Therefore, we prepared a solution with the same concentration of free drug in order to compare the kinetics. A stock solution of 2 mM was prepared by solubilising 2.7 mg of ciprofloxacin in 40 μL of DMSO, diluted to a final volume of 4 ml with 10 mM of PBS (3960 μL). Then 472 μL of the stock solution were diluted to 4 ml with 10 mM of PBS (3528 μL).

As a control, a 200 μM solution of propranolol was prepared as described in the previous section.

The day after depositing the mucus in the donor compartment, we wetted it with 10 μL of milli-q water to balance the hydrogel and waited for 1 hour. Then 200 μL of drug solution were added in the donor compartment, whereas the acceptor compartment was filled with 300 microlitres of 10 mM PBS. Finally, all of the solutions were collected from the acceptor compartments every 15 minutes, 30 minutes, 1 hour, 2 hours, 4 hours, 6 hours and 24 hours and the compartments were filled with 300 μL of 10 mM PBS. The samples were transferred into 1.5 mL Eppendorf® test tubes, diluted to a final volume of 1 mL with 10 mM PBS and analysed by fluorescence. The emission of propranolol was measured at 350 nm (A emission) after excitation at 289 nm (λ excitation); the spectrum was evaluated between 310 nm and 500 nm, with slits 3-4. The emission of ciprofloxacin was measured at 418 nm (A emission) after excitation at 272 nm (λ excitation); the spectrum was evaluated between 290 nm and 500 nm, with slits 3-4.

The intensities were substituted into the calibration curve in order to determine the drug concentrations; based on these data we calculated the mass of the drug in the acceptor compartments (μg), the cumulative mass (μg), the percentage of permeated mass and the percentage of permeated cumulative mass.

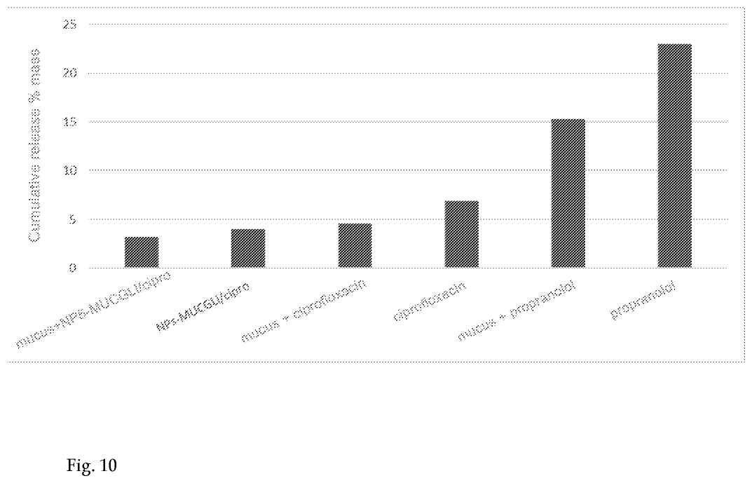

FIG. 10 shows the results obtained.

### Example 5

Quantification of Glycans on the Surface of NPs-MUCGli with PAS Reagent.

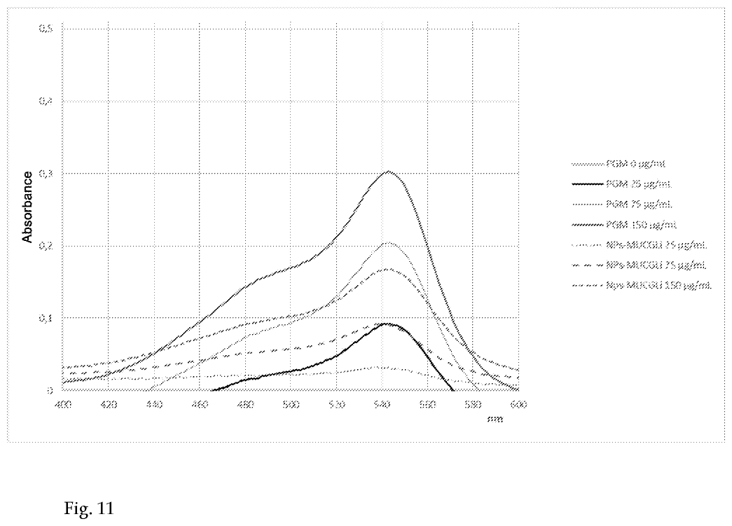

In order to evaluate the actual presence of glycans on the surface of the nanoparticles and be able to quantify them we carried out a colorimetric test with Schiff's reagent. The test is based on a fuchsin-Schiff's reagent reaction. The solution of fuchsin dye is decolorised by the presence of sulphuric acid and turns back to a magenta colour in the presence of glycans, in a manner that is proportional to the concentration of the surface carbohydrates of the NPs-MUCGli. As can be noted from the UV spectrum present in FIG. 11, the amount of surface oligosaccharides highlighted and measured in the NPs-MUCGli is equal to about half the amount present in the starting mucin (PGM). Given an equal concentration expressed in μg/ml, the starting mucin has double the amount of oligosaccharides compared to the NPs-MUCGIi. This means that in the synthesis of the NPs, some oligosaccharides can be “lost”, or else they may not all be exposed on the surface, but rather remain inside the NPs and are thus not quantifiable (in fact, only the oligosaccharides present on the surface of the particles can be quantified).

The data in the presence of ciprofloxacin (NPs-MUCGli/cipro) are comparable to those in the absence of ciprofloxacin (NPs-MUCGli).

### Example 6

Interaction of NPs-MUCGli/FITC with Concanavalin A

In order to demonstrate the ability of the covalently cross-linked glycosylated mucin nanoparticles of the present invention to interact with lectins, a spectrophotometric study was carried out to determine the K_{A} and K_{D} constants of the NPs-MUGGli/FITC-concanavalin A interaction.

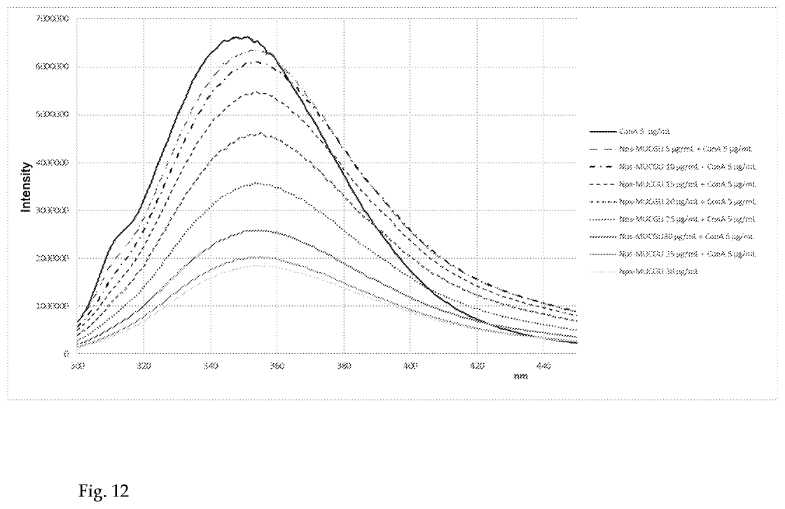

Concanavalin A emits at a wavelength of 350 nm if excited at a wavelength of 280 nm. As may be noted from FIG. 12 the emission intensity decreases (quenching) in the presence of increasing concentrations of porcine gastric mucin nanoparticles loaded with fluorescein.

From an analysis of the data with non-linear fitting (FIG. 12), values of K_{A}=9.1±11×10³M⁻¹ and K_{D}=1.1±0.24M are obtained.

### Example 7

Microbiological Tests—MTT Assay (3-[4,5-dimethylthiazol-2-yl]-2,5-diphenyltetrazolium bromide) for Measuring Enzymatic Activity.

The NPs-MUCGli and the NPs-MUCGli/cipro were prepared in order to perform an MTT test. Before proceeding with the experiments, we conducted a sterility test: the plates were streaked with NPs-MUCGli and NPs-MUCGli/cipro and incubated for 48 hours at 37° C. No contamination was observed.

Then Pseudomonas aeruginosa PAO1 (ATCC 15692) was cultured in Luria Bertani broth (LB) overnight under aerobic conditions at 37° C. in an incubator with orbital shaking and subsequently diluted in LB to obtain a final density of 2×10⁴ cells/mL. The culture was incubated with ciprofloxacin at room temperature and NPs-MUCGli and NPs-MUCGli/cipro.

The nanoparticles were synthesised as described in the previous examples and the following concentrations were determined: the concentration of ciprofloxacin in the NPs-MUCGli/cipro was 1024 μg/mL.

The ciprofloxacin was diluted in LB to a final concentration of 1024 μg/mL, and 100 μL thereof were injected into the first well of the plate, reaching final concentrations respectively of 512 μg/mL of antibiotic in the presence of a suspension of 10⁴ bacterial cells/mL. Starting from the first well, serial dilutions were then performed with a factor of 1:2, the treatment concentration thus being progressively halved up to the sixteenth well. Furthermore, 5 positive controls (the bacterial suspensions not treated with antibiotic), and 5 negative controls (consisting solely of sterile LB) were inoculated.

The experiments on each treatment were carried out in triplicate at least twice. The viability of the bacteria in each well was evaluated via an MTT assay; the survival of the bacteria under each condition was evaluated by comparing the results with those of the respective positive controls.

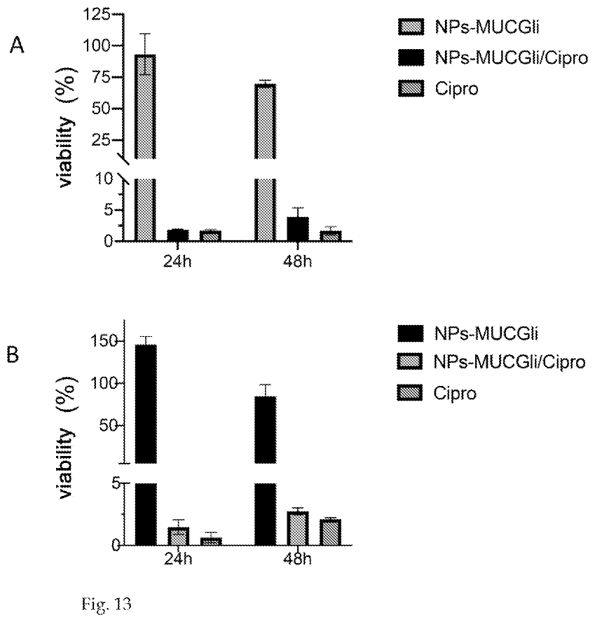

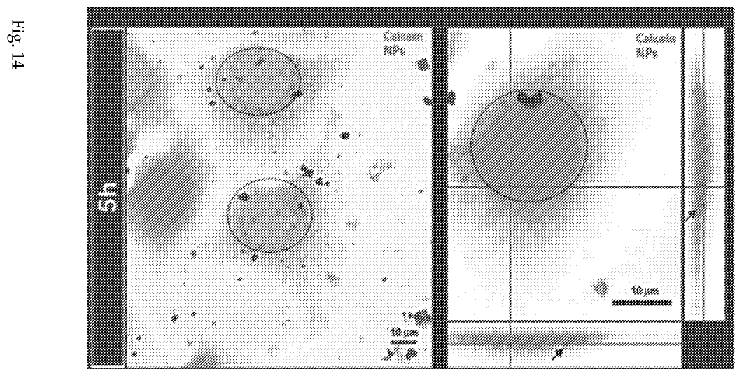

FIG. 13A show the results in terms of % bacterial viability at 24 and 48 hours after treatment with free ciprofloxacin and ciprofloxacin encapsulated in the NPs-MUCGli at a concentration of 2 μg/mL.

Furthermore, the antimicrobial activity of ciprofloxacin, NPs-MUCGli and NPs-MUCGli/cipro was also tested on Staphylococcus aureus (ATCC 25923) cultured in Brain-Heart Infusion (BHI) medium with the same method as described for Pseudomonas aeruginosa (like above, example 7). FIG. 13B shows the results in terms of % bacterial viability at 24 and 48 hours after treatment with free ciprofloxacin and ciprofloxacin encapsulated in the NPs-MUCGli at a concentration of 4 μg/mL. For both bacterial strains it was demonstrated that the activity of the ciprofloxacin is maintained following encapsulation in NPs-MUCGI.

### Example 8

Cellular Internalisation of the Covalently Cross-Linked Glycosylated Mucin Nanoparticles Loaded with FICT (NPs-MUCGli/FITC).

A confocal microscopy study was conducted to assess the cellular absorption of NPs-MUCGli/FITC on HaCaT cells. Before the experiment was set up, the HaCaT cells were seeded onto sterile culture plates and cultured overnight (DMEM 10% FBS). After 24 hours, the 10% FBS DMEM culture medium was replaced by culture medium with a concentration of NPs-MUCGli/FITC of 25 μg/mL.

After the culture plates had been incubated at 37° C. for 5 hours and 20 hours, the modified culture medium was removed. The cells were treated with Calcein AM (CellTrace™, calcein red-orange, Molecular Probe®, Life Technology) to obtain a fluorescent red cytoplasm. The calcein was diluted with HBSS (Hanks' Balanced Salt Solution) at a concentration of 250 nM and then incubated for 30 minutes at 37° C.

Finally, the cells were washed twice with HBSS and fixed at 37° C. with a 4% PFA (paraformaldehyde) solution for 2 minutes. The samples were then ready to be observed by confocal microscopy: in order to visualise them by CLSM (Confocal Laser Scanning Microscopy) a DABCO mounting medium was used. CLSM was performed with a TCS Leica SP8 X (Leica Microsystem) equipped with a scanner with DPSS laser (561 nm, for monitoring the calcein) and Ar laser (488 nm, for monitoring the fluorescein).

The resulting images (1024×1024 pixels or 1152×1152 pixels) were obtained with an oil immersion lens (HC PLAPO CS2 63X/1.4 A.N). A reconstruction of the 3D images helped to understand the adoption of the NPs-MUCGli/FITC. Image J software was used to analyse the images (FIG. 14).

### Example 9—Comparison

Comparison with the Mucin Nanoparticles Obtained in Yan H. Et al. “Reversible Condensation of Mucins into Nanoparticles”, Langmuir (2018), Vol. 34, p. 13615-13625.

The mucin nanoparticles NPs-MUCGli obtained with the method of the invention show significant differences from the reversible nanometric aggregates (PGM YAN) obtained by Yan et. al. First of all, in the preparation of the invention use is preferably made of porcine gastric mucin (PGM) type III rather than directly extracted (native) mucin as in in Yan et al. Native mucin has physicochemical properties that differ slightly from those of commercially available mucin: in fact, it has a greater tendency towards gelation (stabilisation) than commercially available mucin, since native mucin is less pure and less standardised.

In Yan et. al. the authors study the formation of reversible nanometric mucin aggregates in the presence of glycerol in different percentages and H₂O. From the results obtained in Yan et al. it emerges that 30% v/v glycerol/H₂O is the optimal percentage for obtaining these reversible nanometric aggregates. In this document, reversible nanometric aggregates (PGM YAN) are obtained which can be rendered partially stable by adding cross-linkers (polylysine) and calcium.

Starting from the porcine gastric mucin type III used in the present invention (porcine gastric mucin type III) and using the optimal conditions of synthesis described by Yan et al. (30% v/v glycerol/H₂O) we obtained small NPs that were not purifiable by centrifugation even at 4° C. (NPs PGM Yan), which is consistent with the fact that no purification or lyophilisation process for the reversible nanometric aggregates obtained was described in the article.

Furthermore, in Yan et al. no mention was made of glycosylation of the nanometric aggregates obtained.

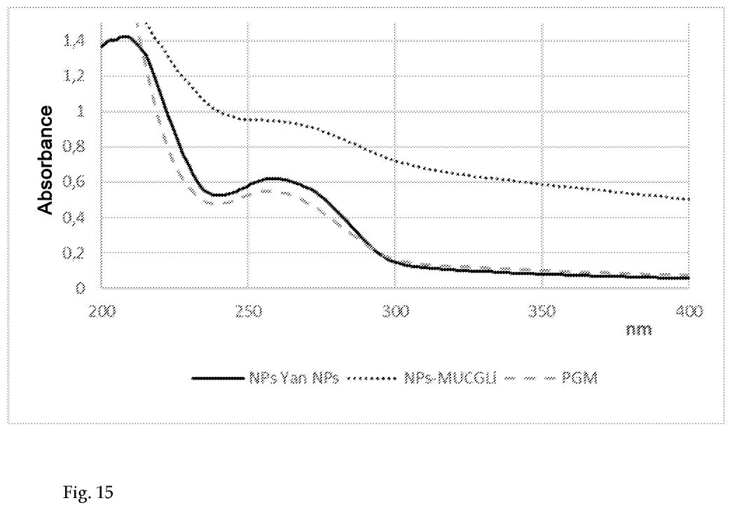

The formation of the NPs PGM Yan with the method described in the literature cannot be monitored by UV: as may be noted from FIG. 15, they maintain substantially the same spectrum as mucin alone, very different from the spectrum of the NPs-MUCGli of the present application.

As regards the characterisation by TEM, it can be noted that the NPs PGM Yan obtained with the method described in the literature do not have well-defined contours, an effect that can also be found in the TEM results reported in the literature for the nanometric aggregates obtained with the native mucin extracted from pig stomach.

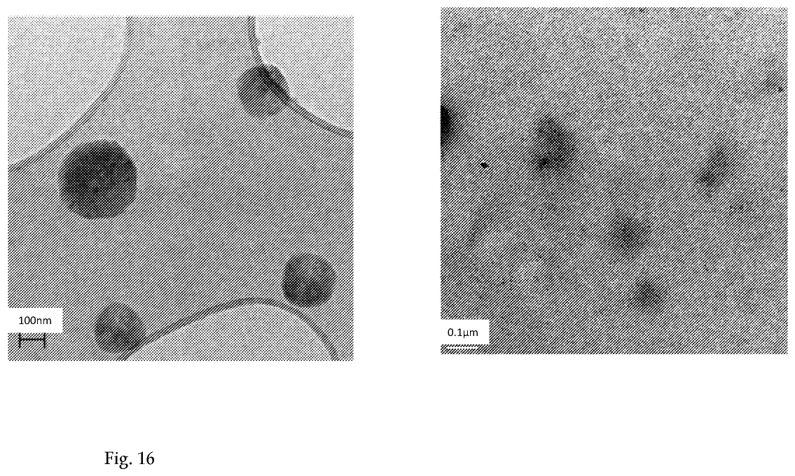

A comparison of TEM images can be seen in FIG. 16: A) NPs-MUCGli obtained from porcine gastric mucin type III with the method described in the present patent application; B) NPs PGM Yan obtained from porcine gastric mucin type III with the method based on glycerol/H₂O described in the literature by Yan et al.

From this comparison it may be inferred that the method of Yan et al., besides not being useful for synthesising stable purifiable and lyophilisable nanoparticles based on native porcine gastric mucin, is not usable even for obtaining stable nanoparticles based on commercial porcine gastric mucin type III, unlike the method proposed in the present invention.

In the cited article, furthermore, no method for incorporating active ingredients, markers and/or biomolecules in the nanoparticles is described, nor is any biological application for the nanometric aggregates obtained described.

### Example 10

Preparation of Covalently Cross-Linked Glycosylated Bovine Submaxillary Mucin Nanoparticles (NPs-MUCGli).

50 mg of bovine submaxillary mucin (BSM) were weighed. Then 2 ml of 10 mM NaCl were added. The solution was left under stirring for 4 hours, and the pH was brought to 8.5 with 0.1 mM NaOH. 8 ml of ethanol were added at 1 gtt/sec. Then 90 μl of 8% glutaraldehyde in milli-q water (cross-linking agent) were added and the solution was left under stirring for 24 hours.

The nanoparticles obtained in solution were purified by centrifugation: they were transferred into a Falcon tube and 5 centrifugation cycles were carried out: 1) 1000 rpm×5 min; 2) 2000 rpm×5 min; 3) 4000 rpm×5 min; 4) 4000 rpm×15 min; 5) 4000 rpm×15 min.

The supernatant was removed at every step and replaced with 1 ml of fresh milli-Q water after every centrifugation cycle.

A solid was obtained from the centrifugation and resolubilised in aqueous solution.

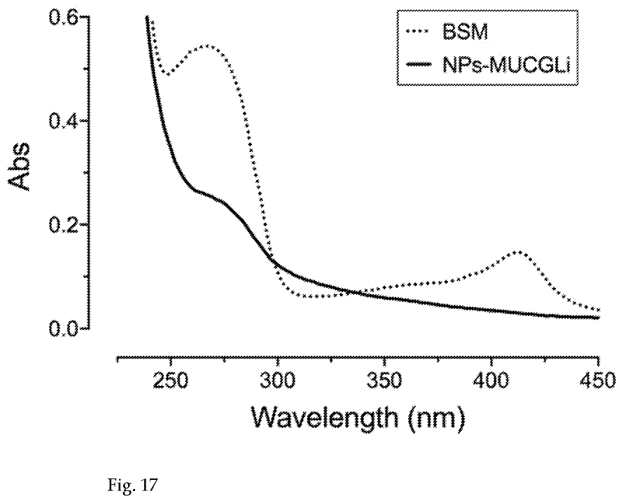

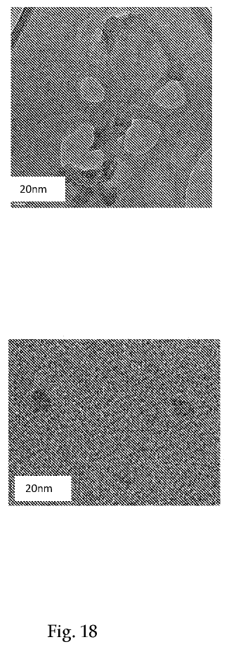

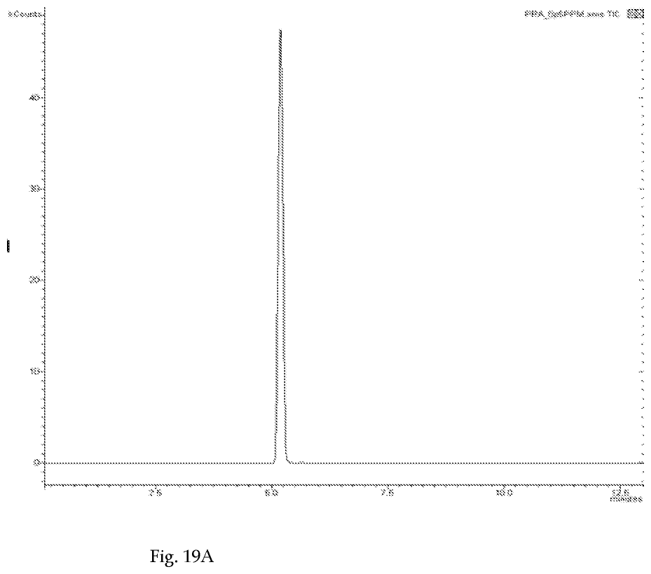

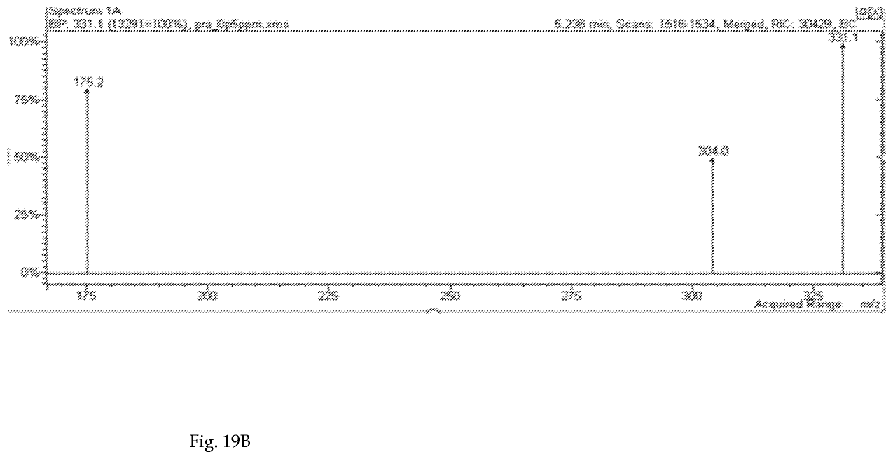

The NPs-MUCGli were lyophilised to obtain the powder form: the sample was divided into three 2 mL aliquots in three different Eppendorf® test tubes and placed in liquid nitrogen until completely frozen. The flask was then connected to a tabletop freeze dryer (HETO LyoLab 3000) equipped with a refrigerator, vacuum centrifuge and vacuum pump to maintain the temperature a −56° C. for a period of 8 hours. The NPs-MUCGli were resuspended in 2 ml of milli-q water and compared with the original sample by means of a UV-Vis study (FIG. 17) and TEM (FIG. 18) and it was observed that the nanoparticles maintain their characteristics.

Characterisation of NPs-MUCGli Nanoparticles

After the synthesis of the NPs-MUCGli a UV-Vis study was conducted: 100 μL of NPs-MUCGIi were diluted to 1 ml with milli-q water (900 μL). The spectrum was measured at 25° C. in the 200-400 nm interval (FIG. 17).

The synthesised nanoparticles were characterised by TEM (model JEOL 3010-UHR) (FIG. 18).

### Example 11

Preparation of Covalently Cross-Linked Glycosylated Porcine Gastric Mucin Nanoparticles Loaded with Active Ingredients with an Antiviral Action: Remdesivir, Camostat, Pralatrexate, RSV 604 (NPs-MUCGli/Remd, NPs-MUCGli/Camo, NPs-MUCGli/Prala and NPs-MUCGli/RSV).

The NPs-MUCGli/remd were synthesised with the same desolvation method as the NPs-MUCGIi of example 1. The NPs-MUCGli/remd were initially characterised by UV-Vis spectroscopy using a concentration of 50 ug/mL.

Pralatrexate, a well-known antitumoural active ingredient was selected, as in a scientific article in the literature it has been described as active against Sars-Cov-2 (Zhang H et al., PLOS Computational Biology, 2020, 1-20).

The NPs containing the active ingredients were prepared by encapsulating the active ingredients using the method described in example 2.

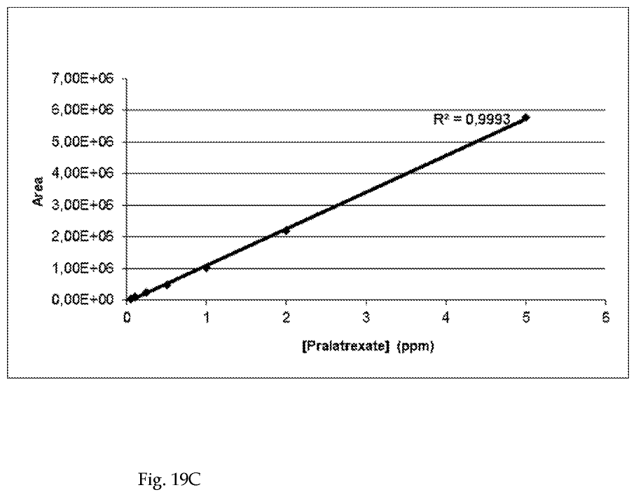

In order to quantify the mass of pralatrexate (MUCGli/prala) encapsulated in the protein nanoparticles, an LC-MS/MS analysis was performed on the supernatants obtained during the purification process (as per example 2). The supernatant from the first wash was diluted 1:100 with milli-q water. A 1:10 dilution was performed for the supernatants of all the other washes. The analysis was carried out using a Varian 320 LC-MS/MS coupled with a Varian 212-LC chromatography system. The characteristics of the method used for the analysis are listed below: Ascentis C18 column, acetonitrile-H₂O as the eluent, 0.1% formic acid, gradient, monitoring the m/z 478>331; 478>304 and 478>175 transitions (FIGS. 19A, 19B and 19C). The % of encapsulation was 15%.

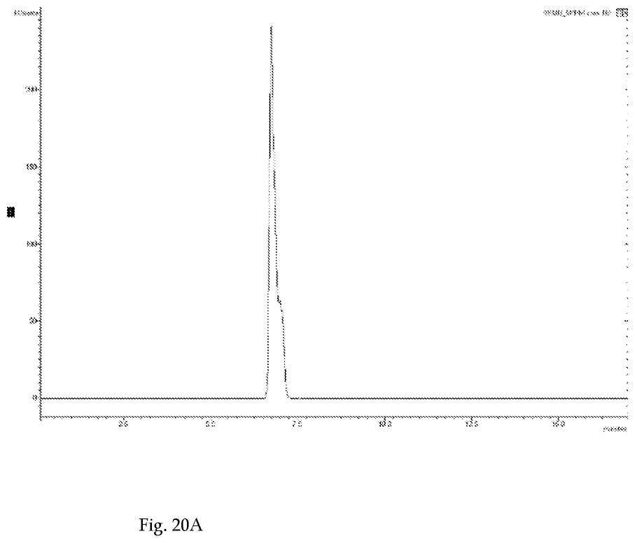

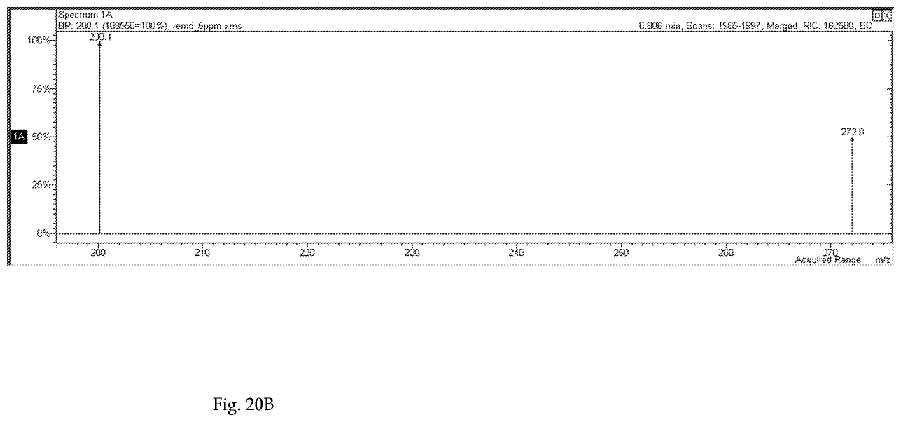

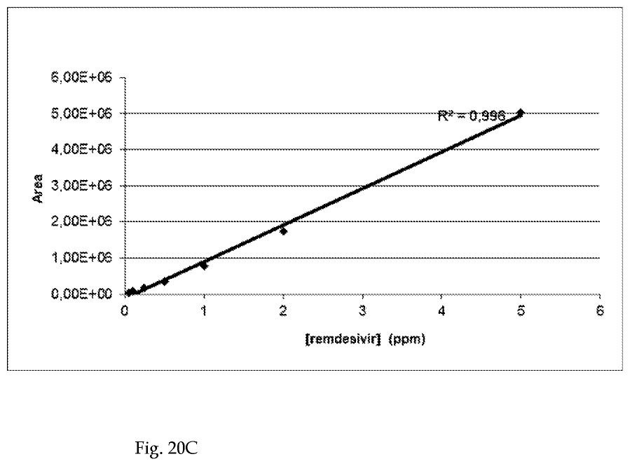

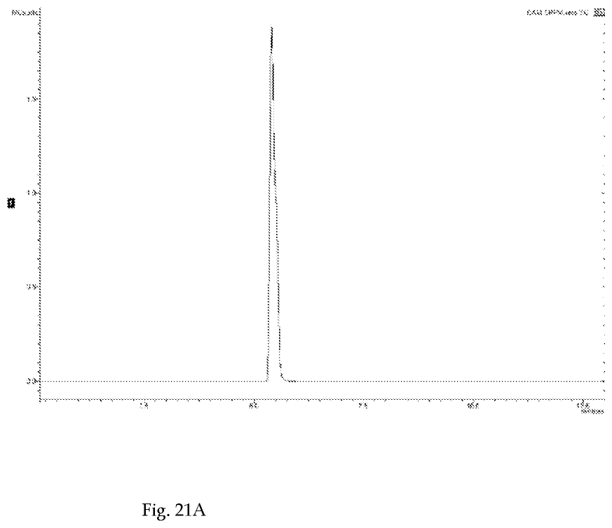

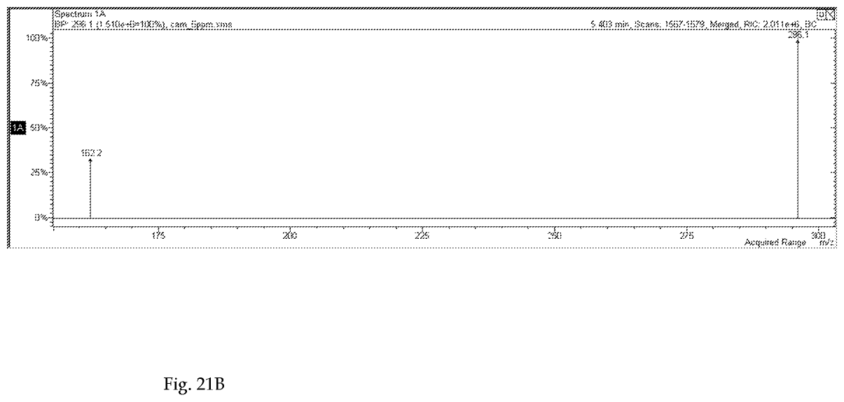

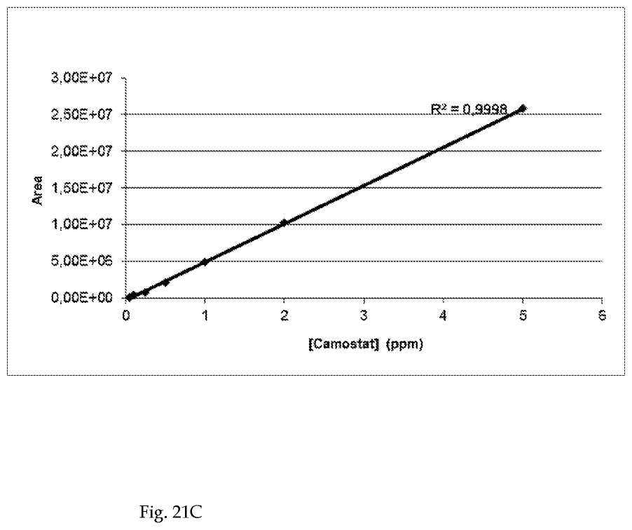

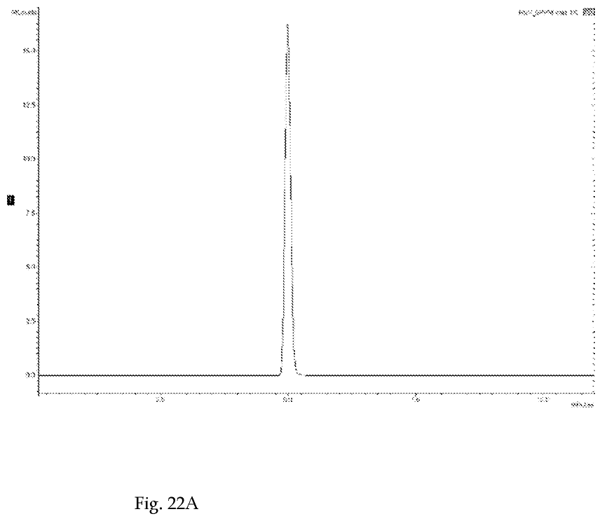

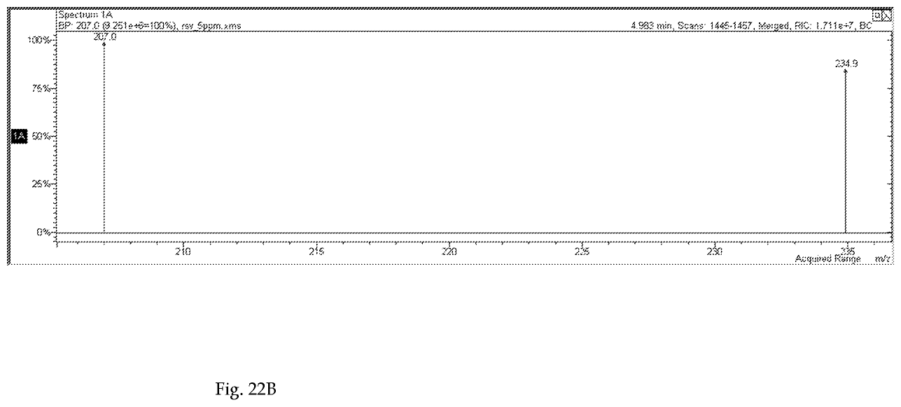

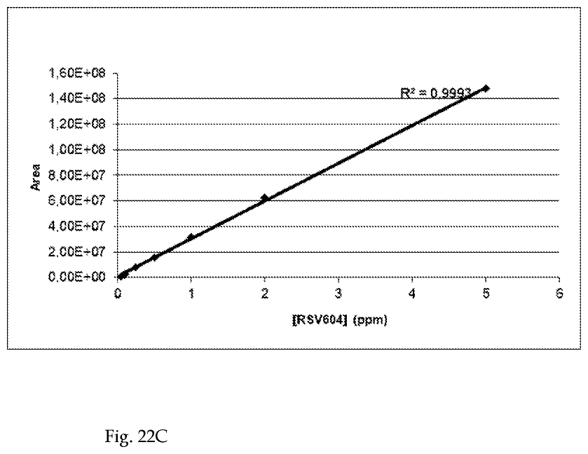

In order to quantify the % of encapsulation of remdesivir, camostat and RSV 604, use was made of the same LC-MS/MS method as used for pralatrexate. In the case of remdesivir the m/z 603>200 transitions were monitored and the % of encapsulation was 10% (FIGS. 20A, 20B and 20C); in the case of camostat the m/z 399>296 transitions were monitored, with a yield of 33% (FIGS. 21A, 21B and 21C); in the case of RSV 604 the m/z 389>207 transitions were monitored with a yield of 80% (FIGS. 22A, 22B and 22C).

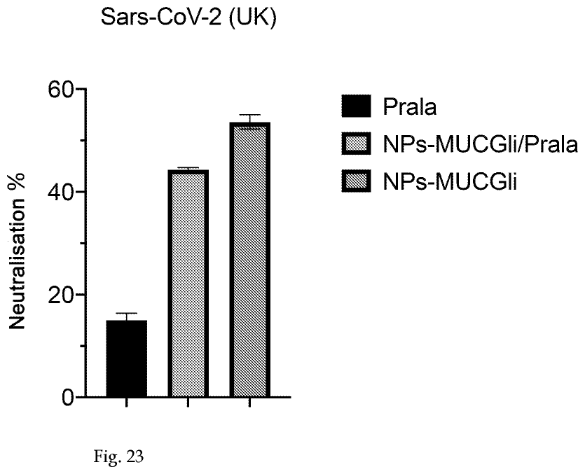

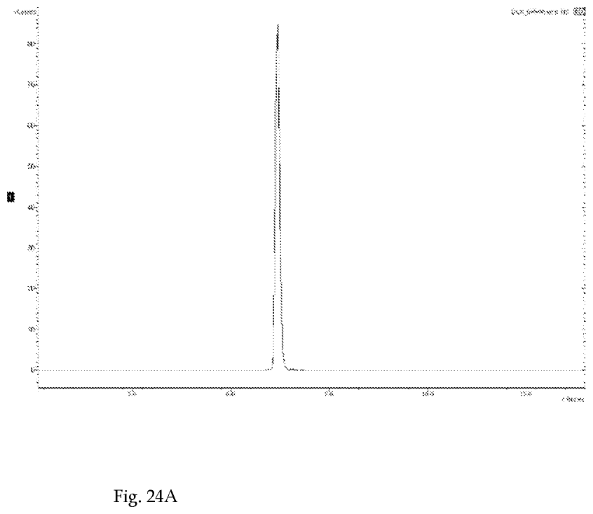

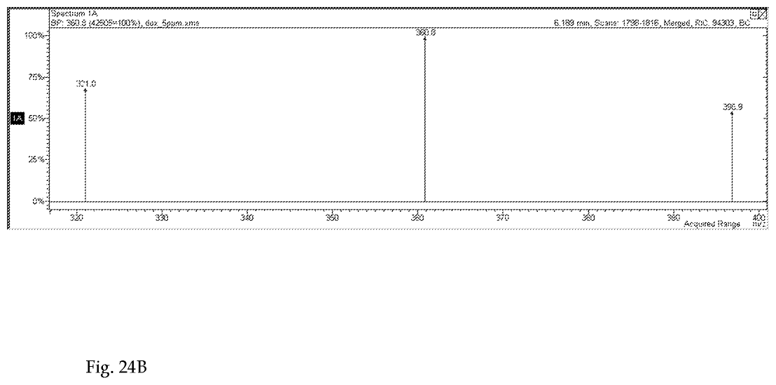

The antiviral activity was evaluated in terms of viral neutralisation in Vero6 cells infected with different variants of Sars-Cov-2. The NPs-MUCGli were tested, both on their own and loaded with the previously described active ingredients. The results indicate that the NPs-MUCGli perform a virus neutralisation activity also in the absence of the active ingredient and the activity of pralatrexate showed to be greater when it was loaded into NPs-MUCGli, as indicated in FIG. 23.

### Example 12

Preparation of Covalently Cross-Linked Glycosylated Porcine Gastric Mucin Nanoparticles Loaded with the Antitumoural Active Ingredients Doxorubicin, Trametinib and the Antitumoural Activity Thereof.

The nanoparticles were synthesised with the same desolvation method as the NPs-MUCGli of example 1.

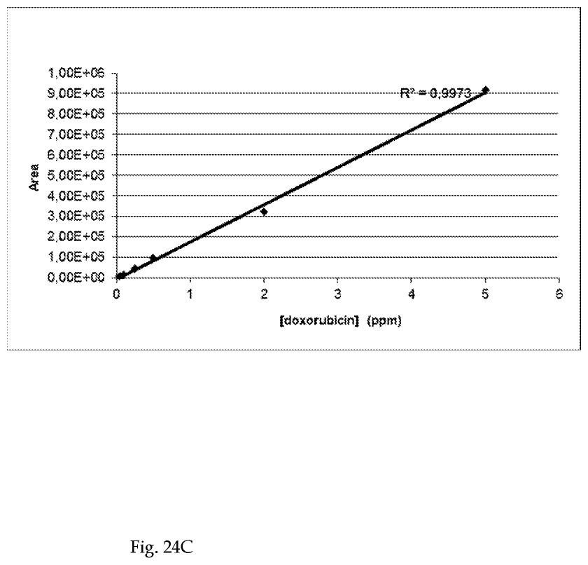

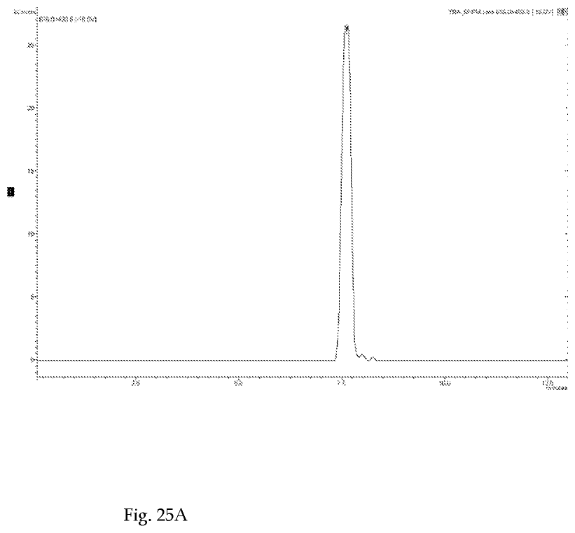

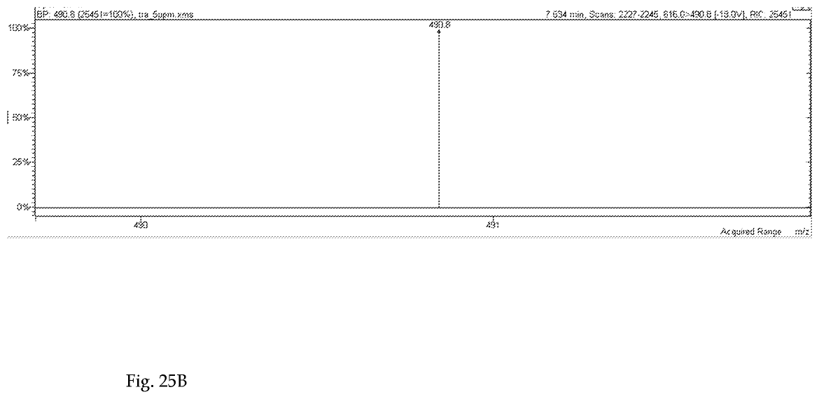

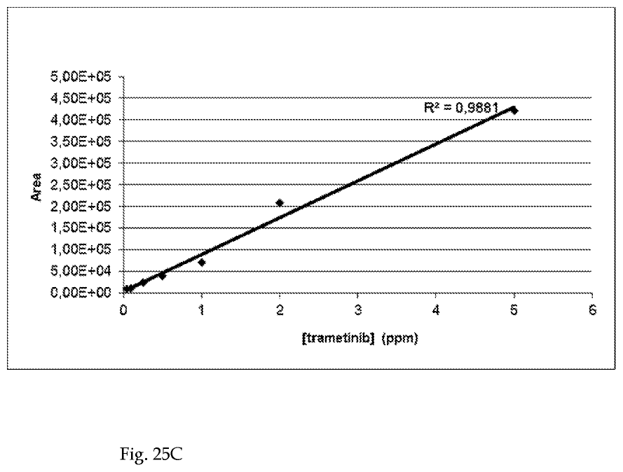

In order to quantify the amount of doxorubicin and trametinib encapsulated in the NPs-MUCGli/doxo and NPs-MUCGli/trame, the LC-MS/MS method described in example 11 was used. In order to measure the encapsulation efficiency for doxorubicin the m/z 544>260 transitions were monitored and the % of encapsulation was 10% (FIGS. 24A, 24B, 24C); in the case of trametinib the m/z 616>490 transitions were monitored and the % of encapsulation was 40% (FIG. 25A, 25B, 25C).

The NPs-MUCGli on their own and with doxorubicin and trametinib were tested on an H358 lung cancer cell line, and the % of cell viability was evaluated in the presence of increasing sample concentrations (Doxorubicin 0.001-10 μM; trametinib 0.001-1 μM).

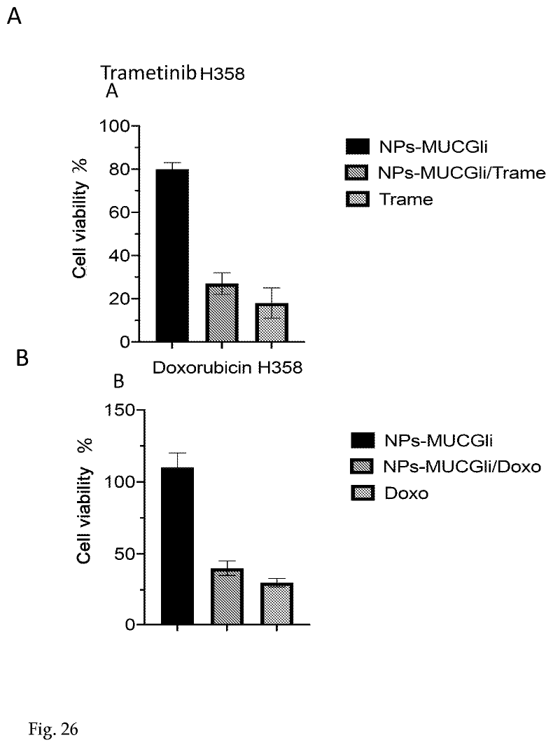

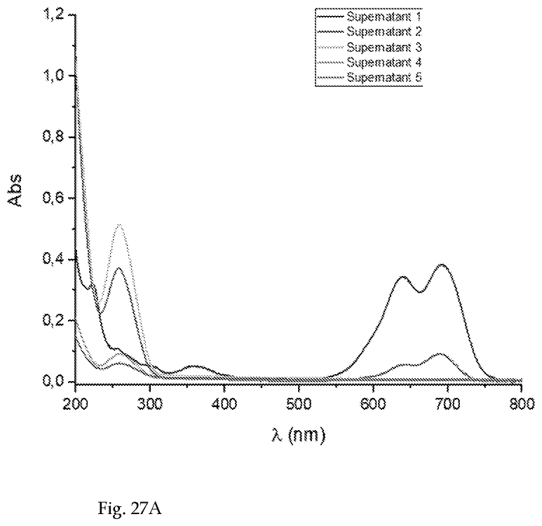

The NPs-MUCGli on their own do not influence cellular activity, thus demonstrating a good cytocompatibility; only when loaded with the active ingredients do they perform their antiproliferative action, without altering the activity of the individual active ingredients. For trametinib we find an 1050 of 7 nM and for NPs-MUCGli/trame an 1050 of 9 nM. In the case of doxorubicin, the 1050 value corresponds to 0.3 nM vs 0.6 for NPs-MUCGli/doxo (FIG. 26).

These data indicate, furthermore, that the amounts of active ingredients present correspond to those measured with the analytical techniques seen previously (FIG. 26).

### Example 13

Preparation of Covalently Cross-Linked Glycosylated Porcine Gastric Mucin Nanoparticles Loaded with the Fluorophore Cyanine 5.5 (NPs-MUCGli/Cy5.5).

The NPs-MUCGli/cy5.5 were synthesised with the same desolvation method as the NPs-MUCGIi of example 1. The NPs-MUCGli/cy5.5 were initially characterised by UV-Vis spectroscopy using a concentration of 50 ug/mL.

Encapsulation Efficiency of NPs-MUCGli/Cy5.5

In order to quantify the amount of cyanine 5.5 encapsulated in the NPs-MUCGli/cy5.5, a UV study was conducted on the supernatants collected during the step of purifying the NPs-MUCGli/cy5.5. 100 μL of supernatant were withdrawn and diluted to 1 ml with milli-q water (900 μL); the spectrum was again measured in the 200-400 nm interval at the maximum absorbance at 760 nm. The concentration of encapsulated fluorophore (μg/mL) was determined by substituting this value into the calibration curve of cyanine 5.5.

Characterisation of NPs-MUCGli/Cy5.5

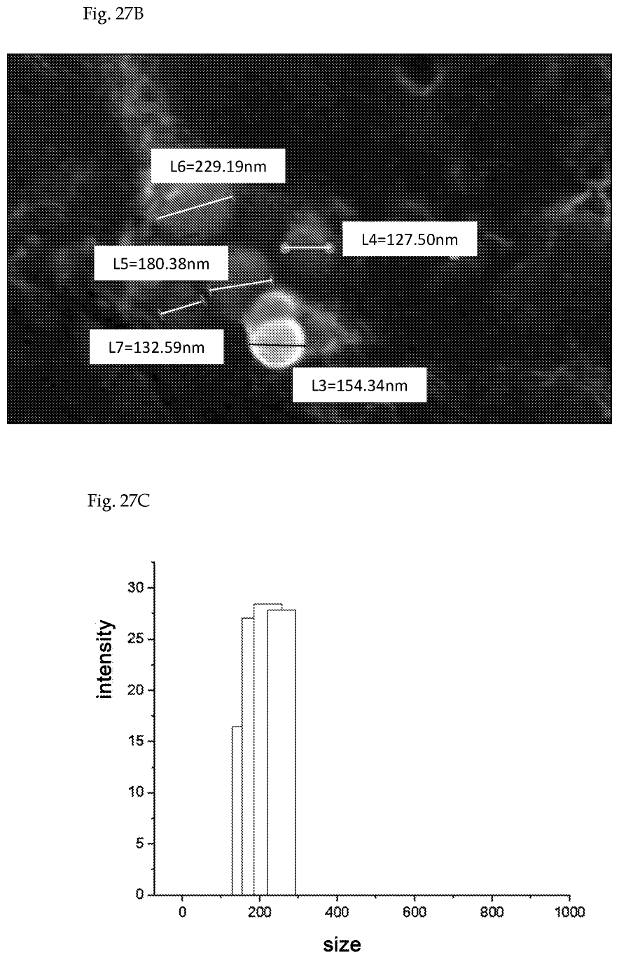

Thanks to Field Emission Scanning Electron Microscopy (FESEM) we were able to monitor the morphology of the NPs-MUCGli/cy5.5: the FESEM images of the sample show that the NPs-MUCGli/cy5.5 are spherical. Based on the FESEM images we established that the average size of the NPs-MUCGli/Cy5.5 is 150 nm. This was confirmed by the data obtained by DLS (FIGS. 27A, 27B and 27C).

Study on Biodistribution and Toxicity In Vivo of NPs-MUCGli/Cy5.5

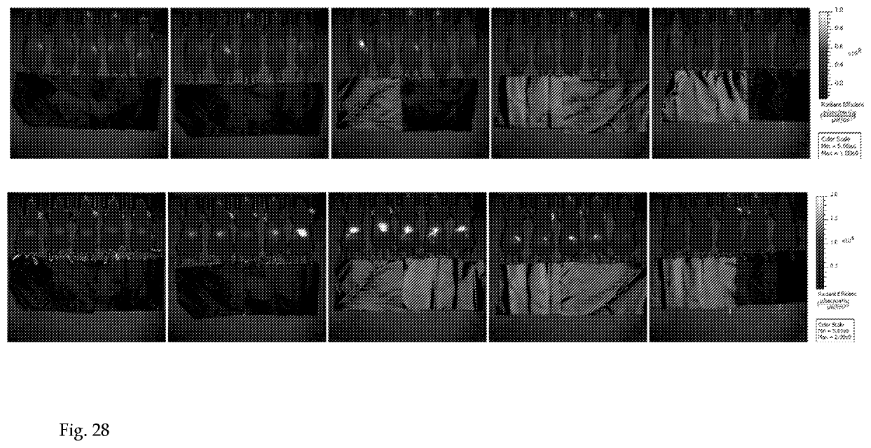

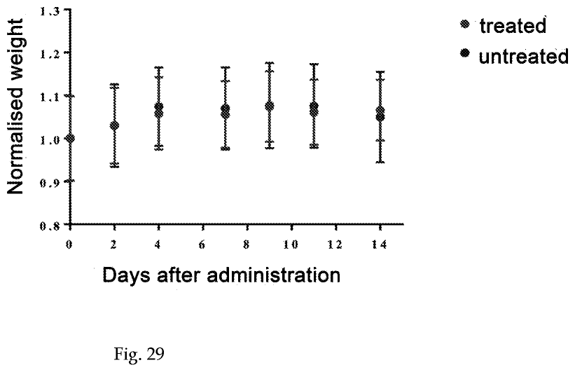

The NPs-MUCGli/cy5.5 5 (100 ug/13 nmol of cy5.5 in 0.25 mL) were injected intravenously into the caudal vein of healthy mice (n=5/group; female athymic nude-foxn1nu 5 weeks) and monitored at 15 minutes, 1 hour, 4 hours, 24 hours and 48 hours. After the injection the animals were anaesthetised with sevoflurane and optical imaging was performed in vivo. After imaging, the animals were sacrificed and the liver, spleen, kidneys, lungs and heart were removed for optical imaging ex vivo (FIG. 28). An intense signal was observed at all of the time points examined. At the earlier times, the strongest signals were found in the liver and lungs, although a significant fluorescent intensity also appeared in the kidneys and gut. A weaker signal was observed in all of the organs analysed 24 hours and 48 hours after the injection. As regards the toxicity studies, NPs-MUCGli/cy5.5 (100 ug in 0.25 mL) were injected intravenously into the caudal vein in five healthy mice; another five mice were treated with 0.25 mL of saline solution. The animals were weighed three times a week and monitored in order to observe the clinical signs (FIG. 29).

### Example 14

Preparation of Covalently Cross-Linked Glycosylated Porcine Gastric Mucin Nanoparticles Loaded with Antimicrobial Active Ingredients: Ceftazidime, Azithromycin (NPs-MUCGli/Cefta, NPs-MUCGli/Azi).

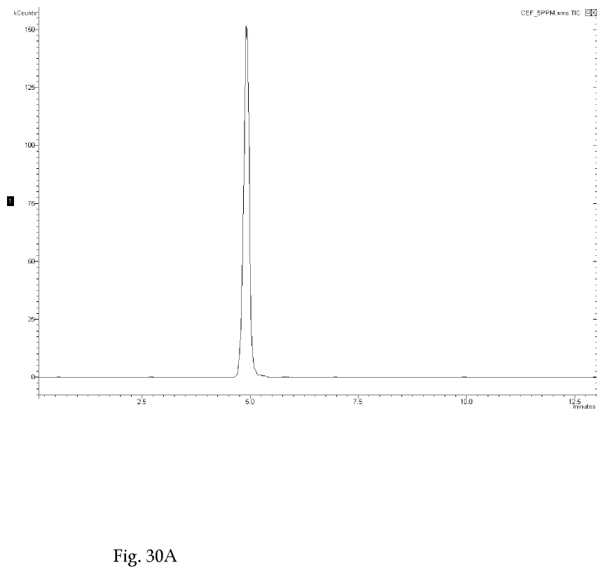

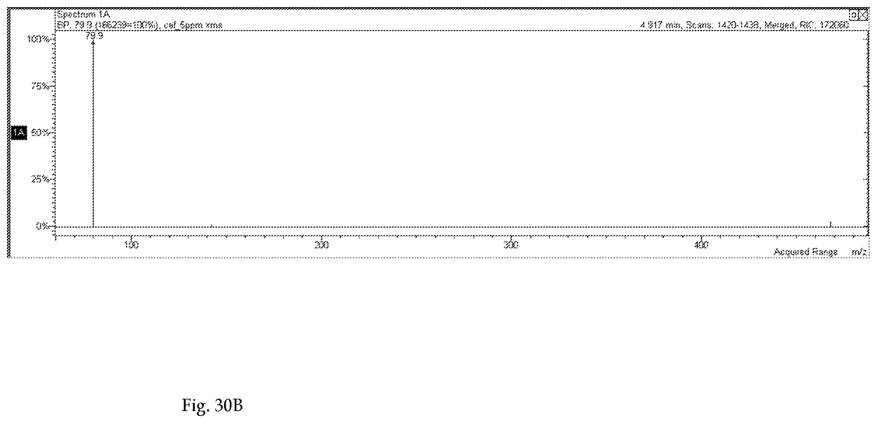

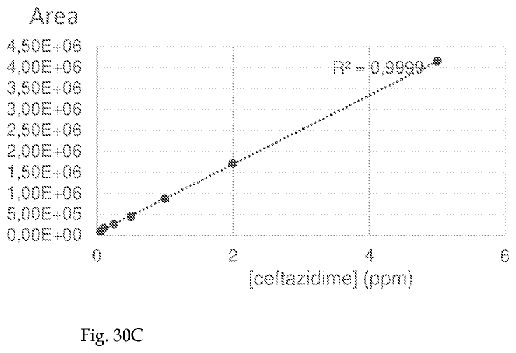

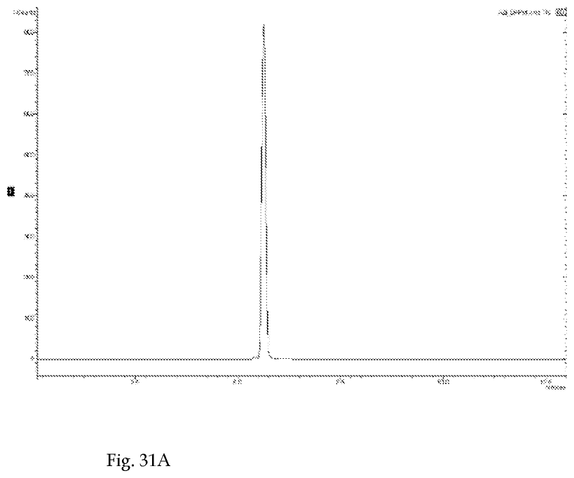

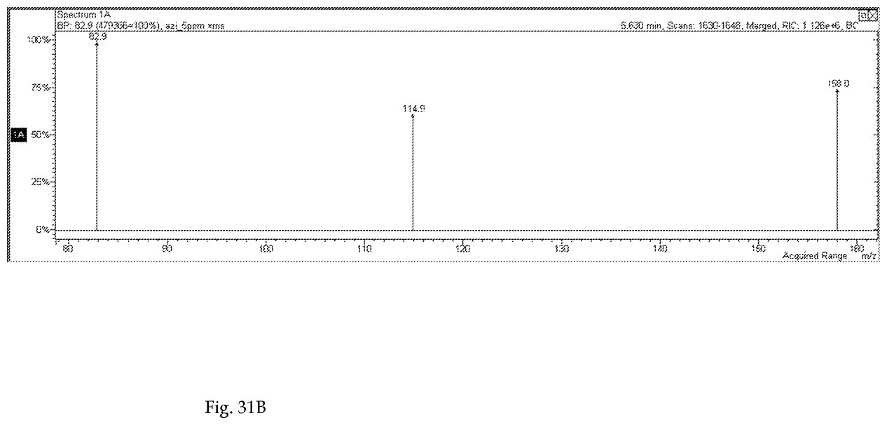

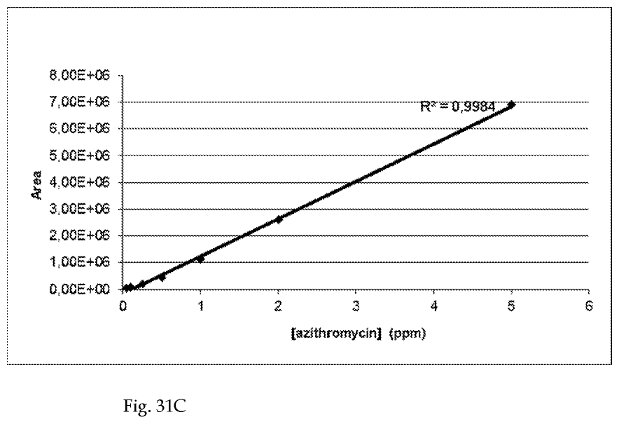

The nanoparticles were synthesised with the same desolvation method as the NPs-MUCGli of example 1. In order to quantify the amount of ceftazidime and azithromycin encapsulated in the NPs-MUCGli, the LC-MS/MS method described in example 11 was used. In the case of ceftazidime, the m/z 274>79 transitions were monitored and the % of encapsulation was 28% (FIGS. 30A, 30B and 30C); in the case of azithromycin, the m/z 375>82 transitions were monitored and the % of encapsulation was 15% (FIGS. 31A, 31B, 31C).

### Example 15

Preparation of Covalently Cross-Linked Glycosylated Porcine Gastric Mucin Nanoparticles Loaded with Anti-Inflammatory Active Ingredients: Dexamethasone, Baricitinib (NPs-MUCGli/Desa, NPs-MUCGli/Bari)

The nanoparticles were synthesised with the same desolvation method as the NPs-MUCGli of example 1. In order to quantify the amount of dexamethasone and baricitinib encapsulated in the NPs-MUCGli, the LC-MS/MS method described in example 11 was used.

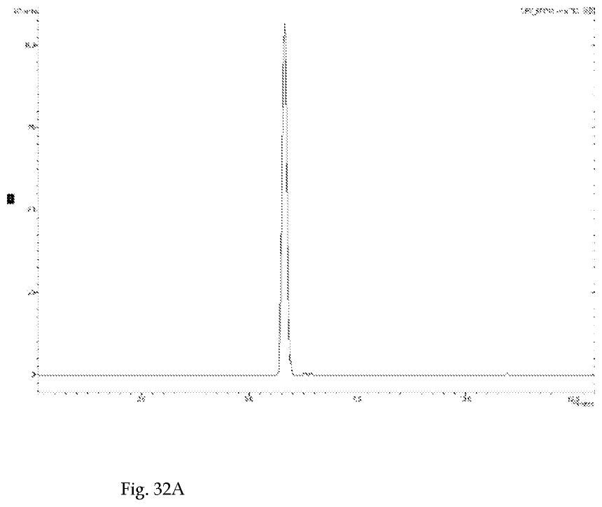

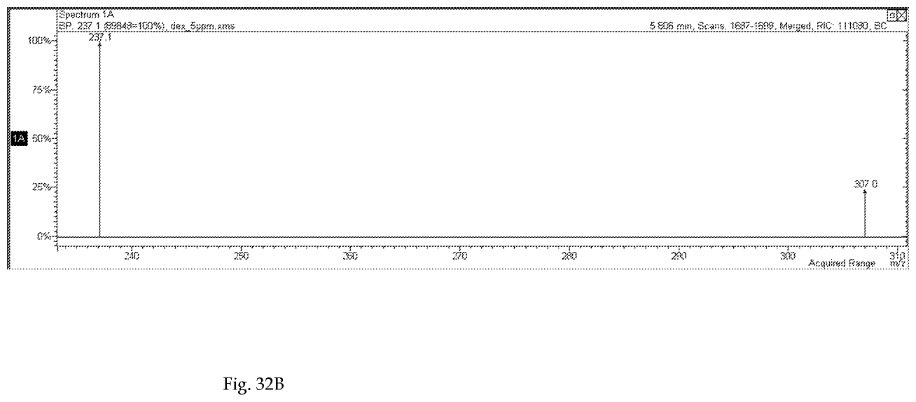

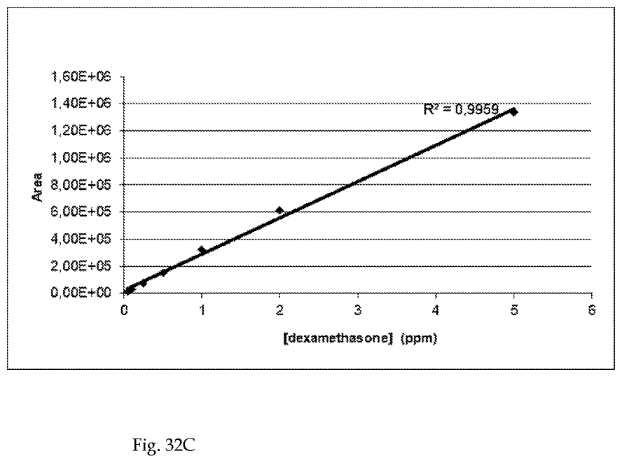

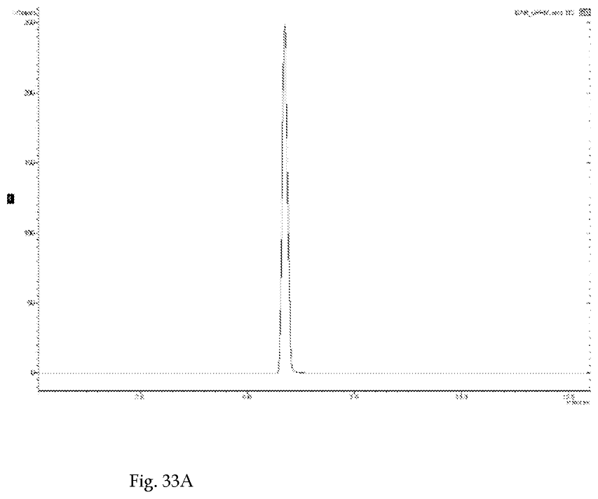

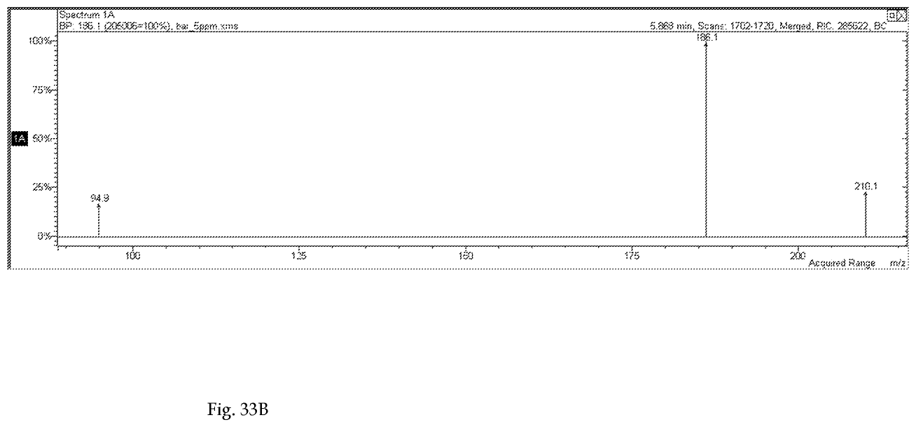

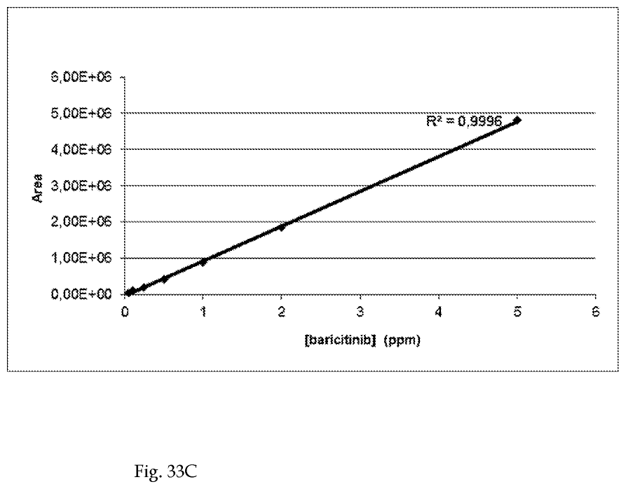

In the case of dexamethasone, the m/z 372>237 transitions were monitored and the % of encapsulation was 20% (FIGS. 32A, 32B and 32C); in the case of barictinib the m/z 371>186 transitions were monitored and the % of encapsulation was 11% (FIGS. 33A, 33B and 33C).

### Example 16

Preparation of Covalently Cross-Linked Glycosylated Porcine Gastric Mucin Nanoparticles Loaded with Albumin-FITC (NPs-MUCGli/Alb-FITC)

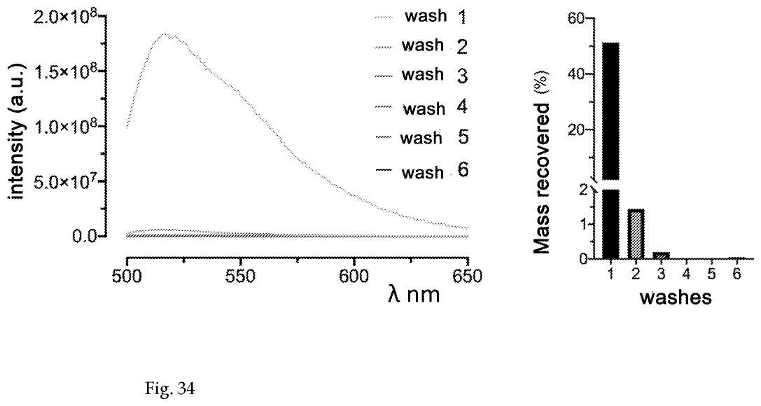

The nanoparticles were synthesised with the same desolvation method as the NPs-MUCGli of example 1. In order to measure the effectiveness of the encapsulation of albumin-FITC the supernatants were analysed by fluorescence and quantified by means of a calibration curve. The encapsulation efficiency in the case of albumin bioconjugated with FITC was 50% (FIG. 34).

### Example 17

Cytotoxicity of NPs-MUCGli

An assessment was made of the toxicity induced by the mucin nanoparticles on HeLa cells. In order to evaluate cell viability, an MTS test was performed. The HeLa cells were seeded at a density of 2.5×10-3 per well in a 96-well multiwell plate and treated with increasing concentrations of mucin nanoparticles for 24, 48 and 72 hours in order to evaluate cell proliferation and viability. Each experiment was conducted three times.

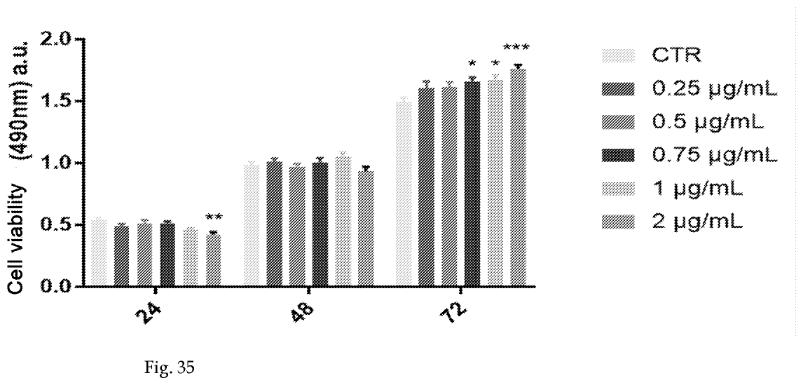

As shown in FIG. 35, the incubation of the NPs-MUCGli did not show any cytotoxic effect in HeLa cells in the range of nanoparticle concentrations compared to untreated HeLa cells (CTRL). The data support a strong biocompatibility of the mucin nanoparticles in vitro vis-à-vis HeLa cells.

### Example 18

Stimulation of the Release of Cytokines in Macrophages in the Presence of NPs-MUCGli

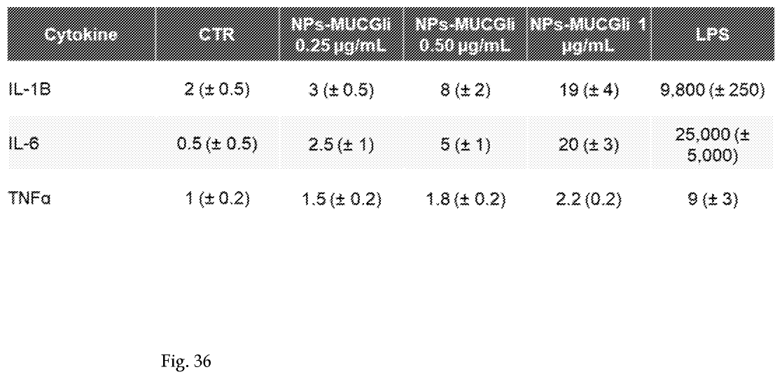

A cell line of mouse macrophages, Raw 264.7, was used to study the cytokine levels after stimulation with NPs-MUCGli. Among the various markers of inflammation, we tested the cDNA levels of the pro-inflammatory cytokines Interleukin-1β (IL-1B), Interleukin-6 (IL-6) and Tumor Necrosis Factor (TNF-α) using a Green Real-Time PCR technique. The cells were treated with NPs-MUCGli at a dose of 1 ug/mL. The untreated cells were used as a negative control. Prior to RNA extraction, the cells were observed by optical microscopy to assess their viability and morphology. The treatment with 0.25 nanoparticles at 1 ug/mL showed no significant toxicity (FIG. 36).

### Example 19

Interaction of NPs-MUCGli with Plasma

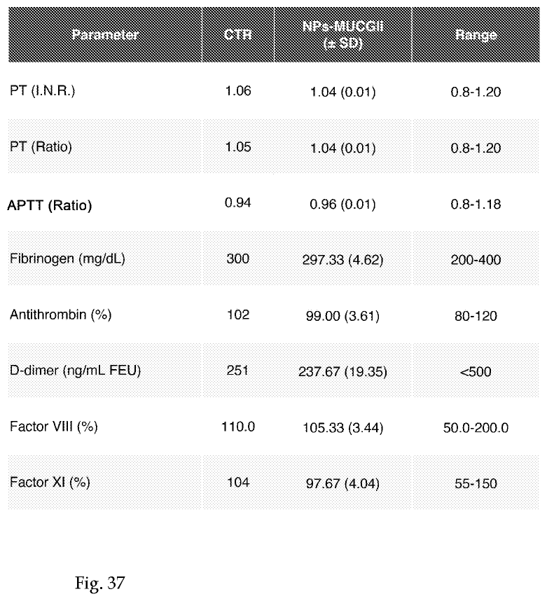

The nanoparticles can also interact with blood, producing aggregation and haemolysis. Blood is in fact the first tissue they come into contact with and it is therefore very important also to understand what the biological response at this level is. For this reason, studies were performed on the primary effects of the NPs-MUCGli on blood coagulation in vitro. The test was performed by adding 3 mg of nanoparticles to blood samples taken from volunteers and an estimate was made of the prothrombin time (PT), the activated partial thromboplastin time (APTT) and the concentration of fibrinogen, antithrombin, D-dimer, factor VIII and factor Xl. As may be noted in FIG. 37 from the comparison of the values obtained in the absence of NPs-MUCGli, it does not seem that the parameters are modified.

### Example 20

Measurement of Mucoadhesion with QCM Microbalance

Mucoadhesion and the interaction with mucus was evaluated using a quartz crystal microbalance (QCM) under flow conditions. Briefly, the sensor, modified with a mucin coating, was placed in a flow cell and exposed to a suspension of NPs-MUCGli. The measurement of the mass adsorbed under flow conditions enabled an evaluation of the interaction with mucin.

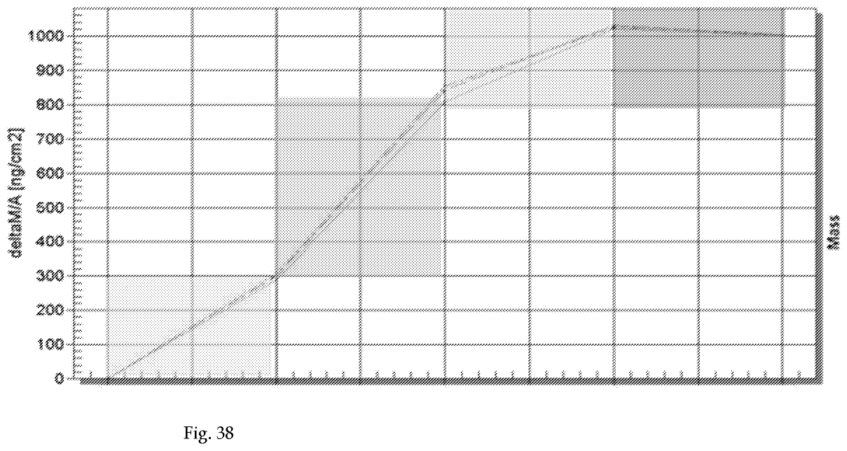

QCM measurements in liquid indicate that with a suspension of NPs-MUCGli (0.25 mg/ml) there is an adsorption of about 200 ng/cm 2 of nanoparticles onto mucin. As may be seen from FIG. 38, the adsorption is stable upon two successive washes with 10 mM PBS (15% decrease in the adsorbed mass). The measurements were made with a gold electrode treated beforehand with a layer of PEI in order to favour the subsequent adhesion of mucin (FIG. 38).

### Example 21

Measurement of the Amount of Sialic Acid

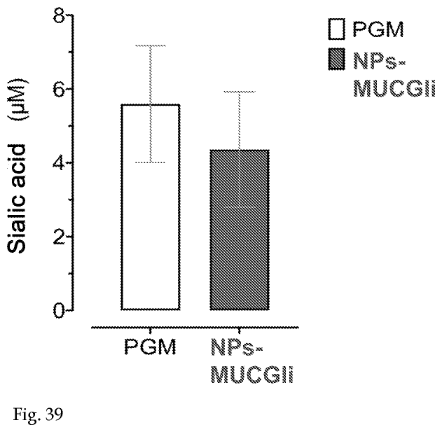

The amount of sialic acid present on the NPs-MUCGli was measured using a kit (Sigma Aldrich Sialic Acid Assay Kit MAK314-1KT) and the measurement was performed on the PGM protein and NPs-MUCGli. As may be seen from FIG. 39, after synthesis a good amount of sialic acid is maintained compared to the starting protein (FIG. 39).

### Example 22

Preparation of Covalently Cross-Linked Glycosylated Porcine Gastric Mucin Nanoparticles Loaded with an Oligonucleotide (NPs-MUCGli/PNA-FITC) The nanoparticles were synthesised with the same desolvation method as the NPs-MUCGli of example 1. An oligonucleotide, preferably a peptide nucleic add bioconjugated with FITC (PNA-FITC), which is a decamer with the sequence TCACTAGATG.

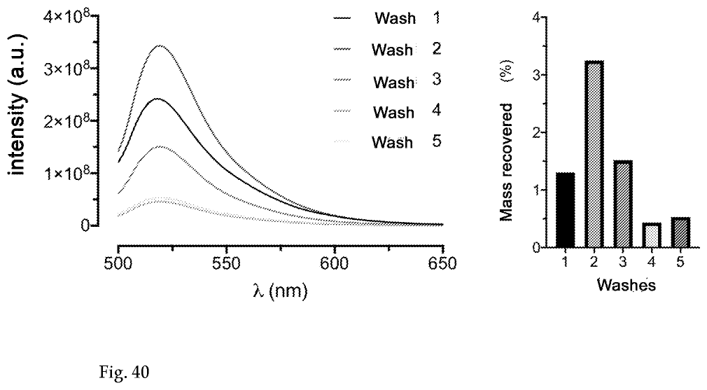

In order to measure the effectiveness of encapsulation of PNA-FITC, the supernatants were analysed by fluorescence and quantified by means of a calibration curve (FIG. 40).

1. Covalently cross-linked glycosylated mucin nanoparticles, wherein the mucin oligosaccharide chains, responsible for glycosylation, are arranged on a surface of the nanoparticles.

2. The covalently cross-linked glycosylated mucin nanoparticles according to claim 1, wherein the mucin oligosaccharide chains are further functionalized with sugars selected from α-L-fucose and one or more compounds of lectin class.

3. The covalently cross-linked glycosylated mucin nanoparticles according to claim 1, wherein the oligosaccharide chains comprise sugars selected from N-acetylgalactosamine, N-acetylglucosamine, fucose, galactose and/or sialic acid.

4. (canceled)

5. The covalently cross-linked glycosylated mucin nanoparticles according to claim 1, comprising at least one compound selected from an active ingredient, a marker and a biomolecule.

6. The covalently cross-linked glycosylated mucin nanoparticles according to claim 5, wherein the active ingredient is an antibiotic selected from aminoglycosides, cephalosporins, quinolones, lincosamides, macrolides, nitroimidazoles, penicillins, sulphonamides, tetracyclines and peptides.

7. The covalently cross-linked glycosylated mucin nanoparticles according to claim 6, wherein the antibiotic belonging to the class of quinolone antibiotics is ciprofloxacin, or wherein the antibiotic belonging to the class of cephalosporins is ceftazidime, or wherein the antibiotic belonging to the class of macrolides is azithromycin.

8. The covalently cross-linked glycosylated mucin nanoparticles according to claim 5, wherein the active ingredient is an antiviral selected from an active ingredient active against influenza viruses, herpes viruses, hepatic viruses, HIV, viruses of the Poxviridae family, the SARS-CoV-2 virus and respiratory syncytial virus.

9. (canceled)

10. The covalently cross-linked glycosylated mucin nanoparticles according to claim 8, wherein the active ingredient that is active against the respiratory syncytial virus is RSV 604.

11. The covalently cross-linked glycosylated mucin nanoparticles according to claim 5, wherein the active ingredient is an antitumoural active ingredient selected from an alkylating agent, an antimetabolite, an antitumoural antibiotic, a topoisomerase inhibitor, a differentiated agent and an immunostimulant.

12. The covalently cross-linked glycosylated mucin nanoparticles according to claim 10, wherein the active ingredient is selected from doxorubicin and trametinib.

13. The covalently cross-linked glycosylated mucin nanoparticles according to claim 5, wherein the active ingredient is an anti-inflammatory active ingredient selected from steroidal and non-steroidal anti-inflammatory active ingredients.

14. The covalently cross-linked glycosylated mucin nanoparticles according to claim 5, wherein the marker is a fluorophore selected from fluorescein isothiocyanate, rose bengal a near-infrared fluorophore and a cyanine.

15. The covalently cross-linked glycosylated mucin nanoparticles according to claim 5, wherein the marker is a biomolecule selected from nucleic acids, peptides, lipids and growth factors.

16-22. (canceled)

23. A “one-pot” method for preparing the covalently cross-linked glycosylated mucin nanoparticles according to claim 1, which comprises the steps of:
a) obtaining a solution of mucin;
a′) optionally adding at least one compound selected from an active ingredient, a marker and a biomolecule to the solution obtained in step a);
b) adjusting the pH of the solution obtained in the preceding step to a pH comprised from 7.5 to 9.5;
c) desolvating the mucin by adding an alcoholic solvent to the solution directly obtained in step b);
d) adding a cross-linker to the solution directly obtained in step c).

24. (canceled)

25. The “one-pot” method according to claim 23, further comprising a step of purifying the covalently cross-linked glycosylated mucin nanoparticles.

26. The “one-pot” method according to claim 23, further comprising a step of lyophilising the purified covalently cross-linked glycosylated mucin nanoparticles.

27. The “one-pot” method according to claim 23, further comprising a step of functionalising the external oligosaccharide chains of the covalently cross-linked glycosylated mucin nanoparticles with sugars selected from α-L-fucose and/or one or more compounds of the lectin class.

28-30. (canceled)

31. A method of treating a viral infection, cystic fibrosis, bronchiectasis, chronic obstructive pulmonary disease, infection caused by the SARS-CoV-2 virus and a pathology involving at least one bacterial strain resistant to at least one antibiotic, said method comprising
administering to a patient in need thereof a pharmaceutically acceptable amount of the covalently cross-linked glycosylated mucin nanoparticles according to claim 5.

32. The “one-pot” method according to claim 25, further comprising a step of purifying the covalently cross-linked glycosylated mucin nanoparticles by centrifugation.
